(19) United States
(12) Patent Application Publication
(10) Pub. No.: US 2020/0253922 A1
(43) Pub. Date: Aug. 13, 2020

(54) METHODS FOR NON-IRRITATING PULMONARY ADMINISTRATION OF CANNABINOIDS USING SOFT MIST INHALERS

(71) Applicant: Molecular Infusions, LLC, Franklin, MA (US)

(72) Inventors: Nicholas J. Boylan, Boylston, MA (US); Scott S. Finnance, Providence, RI (US); Tuna Yucel, Medford, MA (US); Stephen E. Zale, Hopkinton, MA (US)

(21) Appl. No.: 16/788,507

(22) Filed: Feb. 12, 2020

Related U.S. Application Data
(60) Provisional application No. 62/804,905, filed on Feb. 13, 2019.
(60) Provisional application No. 62/804,918, filed on Feb. 13, 2019.

(51) Int. Cl. A61K 31/352; A61K 31/05; A61K 47/10; A61K 47/22; A61K 9/00; A61K 47/02

(52) U.S. Cl. CPC A61K 31/352; A61K 31/05; A61K 47/02; A61K 47/22; A61K 9/0078; A61K 47/10

(57) ABSTRACT

The present invention is directed to the administration of a cannabinoid aerosol formulation using a soft mist inhaler (SMI) with reduced cannabinoid-related irritation and/or coughing. Described is an aerosol formulation comprising a cannabinoid in an ethanol solution and further comprising saccharin or a salt thereof, an aerosol formulation comprising a cannabinoid in a solution comprising ethanol and saline, and methods for the use thereof.

### RELATED APPLICATIONS

This application claims the benefit of U.S. Provisional Application Ser. No. 62/804,905 filed Feb. 13, 2019 and U.S. Provisional Application Ser. No. 62/804,918 filed Feb. 13, 2019. The entire contents of these applications are incorporated by reference herein.

### BACKGROUND OF THE INVENTION

Cannabinoids are a class of active compounds derived from the Cannabis sativa, Cannabis indica, or cannabis hybrid plants commonly known as marijuana. The most notable cannabinoid is the phytocannabinoid tetrahydrocannabinol (THC), the primary psychoactive compound in cannabis. Delta-9-tetrahydrocannabinol ( Δ9-THC) and delta-8-tetrahydrocannabinol (Δ8-THC) mimic the actions of anandamide and 2-arachidonoylglycerol neurotransmitters produced naturally in the body. These cannabinoids produce the effects associated with cannabis by binding to the CB1 cannabinoid receptors in the brain. Cannabidiol (CBD) is another major constituent of the cannabis plant. Other cannabinoids include Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM), Tetrahydrocannabinolic acid (THCA), cannabinol (CBN), and Cannabidiolic Acid (CBDA).

The use of pharmaceutical inhalers or aerosol delivery devices to deliver cannabis-derived therapeutics is an attractive delivery method with notable advantages over more traditional routes of inhalation (e.g., smoking, vaping). Pharmaceutical inhalers offer precise control over dosing and chemical composition, leading to more consistent and reproducible pharmacodynamic effects. However, cough reflex and irritation are major issues limiting the delivery of cannabinoids to the lungs by pharmaceutical inhalers. For example, using a metered dose inhaler, Williams et al. reported that aerosol delivery of 0.2 mg Δ1-THC (now referred to as Δ9-THC) to the lungs of asthmatic patients was tolerable, but that higher doses were irritating and induced coughing [1]. Tashkin et al. reported that a 5 mg dose of Δ9-THC via a pressurized metered dose inhaler (pMDI) resulted in side effects including cough and/or chest discomfort in 3 out of 11 subjects [2]. Naef et al. reported on the pulmonary administration of an aqueous-micellar Δ9-THC formulation using the PARI® nebulizer in eight healthy volunteers at doses greater than 2 mg [3]. Compared to a placebo formulation, all patients reported significant irritation of the throat and upper respiratory tract after inhalation of Δ9-THC. Furthermore, coughing prevented proper inhalation technique which likely contributed to the high inter-patient variability in bioavailability. WO20040002901 described administration of an aerosol formulation comprising Δ8-THC as causing patients to cough and noted that the cough reaction is undesirable as it results in exhalation of much of the inhaled dose.

There remains a need in the art for improved methods and formulations for pulmonary administration of cannabinoids.

### SUMMARY OF THE INVENTION

The present invention is directed to the administration of a cannabinoid aerosol formulation using a soft mist inhaler (SMI) with reduced cannabinoid-related irritation and/or coughing. The invention encompasses the administration of an aerosol formulation comprising a cannabinoid in a solution comprising ethanol, wherein the formulation further comprises saccharin or a salt thereof. The invention also encompasses administration of an aerosol formulation comprising a cannabinoid in a solution comprising ethanol and saline and methods of use thereof. In yet additional aspects, the aerosol formulation comprises a cannabinoid in a solution comprising ethanol and saline, and wherein the formulation further comprises saccharin or a salt thereof. In some embodiments, the soft mist inhaler comprises micron-sized nozzles and/or generates an aerosol by Rayleigh break up.

In certain embodiments, the present invention is based on the discovery that the use of saccharin or a salt thereof in an ethanol-based formulation of a cannabinoid reduces coughing and irritation as compared to an identical formulation in the absence of the saccharin or the salt thereof. The invention is also based on the discovery that the chest discomfort and/or chest tightness experienced by some users after administration of the saccharin-containing formulation can be mitigated by increasing the pH of the formulation to between about 5.5 and about 7.5, for example, by replacing the saccharin with sodium saccharin, while still reducing coughing and irritation.

The invention encompasses a method for pulmonary administration of an aerosol formulation comprising a cannabinoid, the method comprising administering the aerosol formulation to a subject by oral inhalation using a soft mist inhaler, wherein the aerosol formulation comprises the cannabinoid in a solution comprising ethanol, and wherein the formulation further comprises saccharin or a salt thereof, wherein the administration of the aerosol formulation induces less coughing, less throat irritation, and/or less respiratory tract irritation than the administration of an identical formulation in the absence of the saccharin or the salt thereof. In specific aspects, the formulation comprises the salt of saccharin salt, for example, sodium saccharin.

The invention also includes a method for reducing cannabinoid-related irritation and/or coughing of an aerosol formulation comprising a cannabinoid, the method comprising: adding an irritation-reducing amount of saccharin or a salt thereof to an aerosol formulation comprising ethanol and a cannabinoid; loading a soft mist inhaler with the aerosol formulation; and administering the aerosol formulation to a subject by oral inhalation using the soft mist inhaler. In specific aspects, the formulation comprises the salt of saccharin salt, for example, sodium saccharin.

The invention additionally is directed to a method for pulmonary administration of an aerosol formulation comprising a cannabinoid, the method comprising administering the aerosol formulation to a subject by oral inhalation using a soft mist inhaler, wherein the aerosol formulation comprises the cannabinoid in a solution comprising ethanol, wherein the formulation further comprises a saccharin salt and the pH of the formulation is between about 5 and about 7.5, and wherein administration of the aerosol formulation induces less coughing, less throat irritation, and/or less respiratory tract irritation than the administration of an identical formulation in the absence of the saccharin or the saccharin salt. In specific aspects, the formulation comprises the salt of saccharin salt, for example, sodium saccharin.

In certain additional aspects, the invention is an aerosol formulation for pulmonary administration of a cannabinoid by oral inhalation using a soft mist inhaler, wherein the aerosol formulation comprises the cannabinoid in a solution comprising ethanol, and wherein the formulation further comprises saccharin or a salt thereof, wherein the administration of the aerosol formulation comprising the cannabinoid induces less coughing, less throat irritation, and/or less respiratory tract irritation than the administration of an identical formulation in the absence of the saccharin or salt thereof, and wherein the formulation does not comprise a propellant, or a glycol, or a glycol ether. The invention also includes a soft mist inhaler for pulmonary delivery of a cannabinoid, wherein the inhaler comprises a reservoir or cannister for storing the aerosol formulation, and wherein said reservoir or cannister is at least partially filled with said composition.

The invention also includes an aerosol formulation for pulmonary administration of a cannabinoid by oral inhalation using a soft mist inhaler, wherein the aerosol formulation comprises the cannabinoid in a solution comprising ethanol, and wherein the formulation further comprises sodium saccharin and wherein the pH of the formulation is between about 5.5 and 7.5. The invention also includes a soft mist inhaler for pulmonary delivery of a cannabinoid, wherein the inhaler comprises a reservoir or cannister for storing the aerosol formulation, and wherein said reservoir or cannister is at least partially filled with said composition.

In certain aspects, the formulations described herein comprises a saccharin salt, for example sodium saccharin. In certain aspects, the formulation does not comprise a propellant and/or a glycol or a glycol ether.

The present invention is also based on the discovery that the addition of saline to a cannabinoid-containing ethanol-based formulation administered by SMI reduces coughing and irritation and increases the emitted dose of the inhaler as compared to the cannabinoid formulation in the absence of saline. With respect to the effect on emitted dose, as described in the Examples, it was observed that a 200 proof ethanol solution resulted in “sprayback” or caused some of the aerosol to deposit in the mouthpiece of the SMI device. It has been discovered that including saline in the ethanol-based formulation reduces this sprayback effect (reducing the amount aerosol deposited in the mouthpiece). Thus, it was found that the inclusion of saline can increase the emitted dose as compared to that for pure ethanol.

The present invention encompasses a method for pulmonary administration of a cannabinoid aerosol formulation, the method comprising administering the aerosol formulation to a subject by oral inhalation using a soft mist inhaler, wherein the aerosol formulation comprises the cannabinoid in a solution comprising ethanol and saline, wherein the administration of the aerosol formulation induces less coughing, less throat irritation, and/or less respiratory tract irritation than the administration of a control formulation, and/or wherein the emitted dose of the aerosol formulation is greater than that of a control formulation. The control formulation is a formulation identical to the cannabinoid aerosol formulation except that the solution comprises no saline or comprises pure water instead of the saline. The saline can, for example, be present in an amount that induces less coughing, less throat irritation, and/or less respiratory tract irritation than the administration of the control formulation; and/or the saline can be present in an amount that increases the emitted dose of the aerosol formulation as compared to that of a control formulation. In certain aspects, the formulation further comprises saccharin or a salt thereof (for example, sodium saccharin). In certain aspects, the ethanol solution comprising saline comprises about 1% to about 30% by weight saline, or about 5% to about 10% by weight saline, or about 10% by weight saline. In yet further embodiments, wherein the fine particle fraction of the emitted dose of the formulation described herein is at least about 80%.

The invention also includes a method for reducing cannabinoid-related irritation and/or coughing of an aerosol formulation comprising a cannabinoid, the method comprises including an irritation-reducing amount of saline in an aerosol formulation comprising ethanol and a cannabinoid and loading a soft mist inhaler with the aerosol formulation. In certain aspects, the formulation further comprises saccharin or a salt thereof (for example, sodium saccharin). In certain aspects, the ethanol solution comprising saline comprises about 1% to about 30% by weight saline, or about 5% to about 10% by weight saline, or about 10% by weight saline. In yet further embodiments, wherein the fine particle fraction of the emitted dose of the formulation described herein is at least about 80%.

The invention additionally encompasses a method for increasing the emitted dose of the aerosol formulation using a soft mist inhaler as compared to that of a control formulation, the method comprising including saline in the aerosol formulation comprising ethanol and a cannabinoid and loading a soft mist inhaler with the aerosol formulation, wherein the saline is added in amount that increases the emitted dose. In certain aspects, the formulation further comprises saccharin or a salt thereof (for example, sodium saccharin). In certain aspects, the ethanol solution comprising saline comprises about 1% to about 30% by weight saline, or about 5% to about 10% by weight saline, or about 10% by weight saline. In yet further embodiments, wherein the fine particle fraction of the emitted dose of the formulation described herein is at least about 80%.

In addition, the invention is directed to an aerosol formulation for pulmonary administration of a cannabinoid by oral inhalation using a soft mist inhaler, wherein the aerosol formulation comprises the cannabinoid in a solution comprising ethanol and saline, wherein the administration of the aerosol formulation induces less coughing, less throat irritation, and/or less respiratory tract irritation than the administration of a control formulation, and/or wherein the emitted dose of the aerosol formulation is greater than that of a control formulation; wherein the control formulation is a formulation is identical to the cannabinoid aerosol formulation except that the solution comprises no saline or comprises pure water instead of the saline. The invention also includes a soft mist inhaler comprising a reservoir or cannister for storing the aerosol formulation described herein and wherein said reservoir or cannister is at least partially filled with said formulation.

The invention also includes a method for pulmonary administration of an aerosol formulation comprising a terpene, the method comprising administering the aerosol formulation to a subject by oral inhalation using a soft mist inhaler, wherein the aerosol formulation comprises the terpene in a solution comprising ethanol, and wherein the formulation further comprises saccharin or a salt thereof, wherein the administration of the aerosol formulation induces less coughing, less throat irritation, and/or less respiratory tract irritation than the administration of an identical formulation in the absence of the saccharin or the salt thereof. In specific aspects, the formulation comprises the salt of saccharin salt, for example, sodium saccharin.

The invention also includes a method for reducing terpene-related irritation and/or coughing of an aerosol formulation comprising a cannabinoid, the method comprising: adding an irritation-reducing amount of saccharin or a salt thereof to an aerosol formulation comprising ethanol and a terpene; loading a soft mist inhaler with the aerosol formulation; and administering the aerosol formulation to a subject by oral inhalation using the soft mist inhaler. In specific aspects, the formulation comprises the salt of saccharin salt, for example, sodium saccharin.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a plot of viscosity (cP) as a function of the percentage (%) w/w of Δ9-THC distillate, or CBD isolate in 200 proof ethanol.

FIGS. 2A and 2B are plots of percent dose of Δ9-THC in the fine particle fraction (FPF) as a function of viscosity (cP) (FIG. 2A) and emitted dose (mg) (FIG. 2B). Formulations with viscosity less than 2 cP and/or less than 2 mg Δ9-THC delivered about 80% of the dose in FPF.

FIG. 3 is a schematic showing the conversion of acidic saccharin to sodium saccharin by the addition of sodium hydroxide.

FIG. 4 is a plot showing microspecies distribution as a function of cannabinoid formulation pH.

FIG. 5 is a photograph of 2% w/w Δ9-THC and 2% w/w CBD in solutions of 10%, 20%, 30%, 40% and 50% (w/w) saline in ethanol. Δ9-THC and CBD were soluble in solutions of up to 30% and 40% (w/w) saline, respectively. In 40% and 50% (w/w) saline solutions, Δ9-THC was observed to form a separate liquid phase (oiling out). In 50% (w/w) saline solution, CBD was observed to precipitate.

FIG. 6 is an illustration depicting the “sprayback” observed using 200 proof ethanol solution in the MEDSPRAY® Inhaler device as compared to the direction of aerosol observed using a 5% by weight saline in ethanol solution.

### DETAILED DESCRIPTION OF THE INVENTION

A description of preferred embodiments of the invention follows.

As used herein, the words “a” and “an” are meant to include one or more unless otherwise specified. For example, the term “a cannabinoid” encompasses both a single cannabinoid and a combination of two or more cannabinoids.

Unless otherwise indicated, all numbers expressing reaction conditions, quantities of ingredients, and so forth, as used in this specification and the claims are to be understood as being modified in all instances by the term “about.”

The term “respiratory tract” means the part of the anatomy of the respiratory system involved with the process of respiration and includes the upper respiratory tract and the lower respiratory tract. The term “upper respiratory tract” refers to the nasal cavity, oral cavity, pharynx, and larynx. The term “lower respiratory tract” refers to the trachea, main bronchi, lobar bronchi, segmental bronchi, subsegmental bronchi, conducting bronchioles, terminal bronchioles, and lungs, which include the, respiratory bronchioles, alveolar ducts, alveolar sacs, and alveoli.

As used herein, when a range is set forth as “between” two values, it is understood that the range is inclusive of the end values.

As used herein, the terms “treat”, “treating” or “treatment” means to alleviate, reduce or abrogate one or more symptoms or characteristics of a disease, disorder or event, cause a desired biological effect, and/or may be curative, palliative, prophylactic or slow the progression of the disease or disorder. For example, treatment can include reducing or eliminating irritation in the throat or respiratory tract of an individual and/or reducing, avoiding or eliminating cough. Additionally, treatment can include achieving a psychoactive effect in an individual.

The term “effective amount” means an amount of active ingredient(s) that will result in a desired effect or result. The term “therapeutically effective amount” means an amount of active ingredient(s) that will elicit a desired biological or pharmacological response, e.g., effective to prevent, alleviate, or ameliorate symptoms (e.g., reducing or eliminating irritation and/or coughing and/or respiratory tract irritation), treat a disease or disorder (e.g., nausea); or cause a psychoactive effect in the individual.

The term “patient” or “subject” means an animal, including mammals, non-human animals, and especially humans. In one embodiment, the patient or subject is a human. In another embodiment, the patient or subject is a human male. In another embodiment, the patient or subject is a human female. The patient can be a healthy individual or an individual in need of medical treatment. In particular, the term “patient” is intended to include individuals that can medically benefit from the administration of a cannabinoid as well as individuals who can benefit recreationally.

As used herein, “surfactant” refers to synthetic and naturally occurring amphiphilic molecules that have hydrophobic portion(s) and hydrophilic portion(s). Due to their amphiphilic (amphipathic) nature, surfactants typically can reduce the surface tension between two immiscible liquids, for example, the oil and water phases in an emulsion, stabilizing the emulsion. Surfactants can be characterized based on their relative hydrophobicity and/or hydrophilicity. For example, relatively lipophilic surfactants are more soluble in fats, oils and waxes, and typically have HLB values less than or about 10, while relatively hydrophilic surfactants are more soluble in aqueous compositions, for example, water, and typically have HLB values greater than or about 10. Relatively amphiphilic surfactants are soluble in oil- and water-based liquids and typically have HLB values close to 10 or about 10.

Total emitted dose or delivered dose (TED) is the mass of drug emitted per actuation that is available for inhalation at the mouth. The total emitted dose can be measured using a dose uniformity sampling apparatus. Fine particle dose (FPD) is the mass of particles less than 5 microns in size within the total emitted dose. Fine particle fraction (FPF) is the fine particle dose divided by the total emitted dose. Coarse particle fraction is the proportion of particles in the total emitted dose that are greater than 5 microns in size. These properties can be measured as described below in the working examples using known equipment, including a cascade impactor.

The terms “total emitted dose” and “emitted dose” are used interchangeably herein.

“Pharmaceutically acceptable salts,” or “salts,” include the salt of a cannabinoid (including, for example, a cannabinoid prodrug or a cannabinoid synthetic analog that includes a basic group) suitable for administration to a mammal, including those prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric, galacturonic, hydrochloric, hydrobromic, sulfuric, nitric, and phosphoric acids.

As discussed above, the present invention is directed to the improved administration of a cannabinoid to a subject using a soft mist inhaler (SMI), such as SMIs comprising micron-sized nozzles and/or that generate an aerosol (or monodisperse droplets) by Rayleigh break up, as well as methods of reducing cough and/or respiratory tract irritation induced by pulmonary administration of a cannabinoid. Soft mist inhalers generate a ‘soft mist’ or liquid aerosol by mechanical, thermo-mechanical or electrochemical means and enable the use of propellant-free drug solutions. In certain embodiments, the soft mist inhaler generates a soft mist or liquid aerosol by mechanical means. The term “soft mist” describes both the mechanism of aerosol generation and the characteristics of the aerosol formed (Anderson, 2006. International Journal of COPD 1(3): 251-259). As compared to pressurized meter dose inhaler (pMDI) and dry powder inhalers, the emitted aerosol of a soft mist inhaler is lower velocity and has a longer spray duration which results in higher percentage of emitted dose being deposited in the lungs. A non-limiting example of a soft mist inhaler is the RESPIMAT® Soft Mist Inhaler which generates fine aerosols for inhalation by forcing the drug formulation through a uniblock assembly that generates two converging jets of solution which collide at a controlled angle (Dalby et al. (2011), Med Devices (Auckl) 4: 145-155; U.S. Pat. No. 7,571,722; U.S. Pat. App. Pub. No. 2012/0103326, WO2005/079997A1, WO97/12687, and WO94/07607 each incorporated by reference herein in their entireties). Exemplary properties of the RESPIMAT® soft mist inhaler are presented below in Table A (Dalby et al.):

TABLE A

| Property | Value | |
|---|---|---|
| Metering Chamber | 15 | ul |
| Spray duration | 1.5 | s |
| Spray velocity | 0.8 | m/s |
| Static force | 45 | N |
| Transient Pressure | 25 MN/m² (250 bar) | |
| Actuations | 60 or 120 | |
| Torque to cock device | 40 | cNm |

Another soft mist inhaler is the MEDSPRAY® Inhaler device which consists of an outer housing, reservoir/cannister, internal pump and spray nozzle chip that contains approximately 100 micron-size nozzles. The liquid aerosol formulation is dispersed into droplets by forcing the drug solution through an array of nozzles with mechanical means which results in generation of Rayleigh jets that break-up into micron size particles (jet break up due to capillary instability). Incorporation of an air-mixing chamber allows the micron size particles to mix with the air stream during the patient's inhalation maneuver. Such devices are described, for example, in de Boer et al. (2008), Pharmaceutical Research 25(5): 1186-1192; Wissink et al. (2002), Respiratory Drug Delivery available at https://www.researchgate.net/publication/267721675. In certain embodiments, the soft mist inhaler is a soft mist inhaler comprising micron-sized nozzles and/or that generate aerosol (or monodisperse droplets) by Rayleigh break up.

The AERx™ aerosol delivery system is also an example of a soft mist inhaler. This device utilizes a blister package containing a single unit dose reservoir and an array of 2.5 micron size nozzles. During actuation, the reservoir is pressurized and a seal is ruptured allowing formulation to flow to the nozzles. As the drug formulation exits the nozzles, a liquid jet is formed which spontaneously breaks up into liquid droplets which exit the devices in the form of an aerosol. See e.g., U.S. Pat. Nos. 5,622,162A and 5,522,385, incorporated by reference in their entireties.

In certain embodiments, the inhaler is a soft mist inhaler comprising micron-sized nozzles and/or that generate an aerosol (or monodisperse droplets) by Rayleigh break up. Specific examples of such inhalers that comprise micron-sized nozzles are the MEDSPRAY® Inhaler device and the AERx™ aerosol delivery system described above. An example of a device that comprises micron-sized nozzles and generates an aerosol by Rayleigh break up is the MEDSPRAY® Inhaler device.

The MEDSPRAY (MIST Beta) inhaler device was designed to provide an airflow resistance of −2 kPa at a flow rate of 15 L/min. Utilizing inhaler devices with decreased airflow resistance (at a fixed inhalation flow rate) can under certain circumstances enable users to inhale at higher inspiratory flow rates with less overall effort and result in improved user experience. In certain embodiments, the inhaler device used according to the methods described herein has an Airflow Resistance at a flow rate of 15 L/min of about −2 kPa or less, about −1.5 kPa or less, about −1 kPa or less, or about −0.5 kPa or less. In yet additional aspects, the inhaler device used according to the methods described herein has an Airflow Resistance less than about −2 kPa at a flow rate of 15 L/min. Airflow Resistance value is useful for assessing the inspiratory effort required by users to achieve a desired inhalation flow rate through a medical inhaler. Airflow Resistance measurements can be performed using a custom experimental set-up consisting of a Copley Scientific's Dry Powder Inhaler dose uniformity sampling apparatus (DUSA) connected to a TPK 2000 critical flow control and in-line DFM 2000 flow meter followed by LCPS Diaphragm vacuum pump. Devices can be attached to the DUSA inlet via silicone mouthpiece adapter and airflow can be initiated by turning on LCPS pump. To assess appropriate flow rates for in vitro characterization of an inhaler device, the flow rates achieved by human users can be measured by attaching a flow meter to mouthpiece body via rubber stopper adapter. Users inhale through the device with minimal, moderate, and high effort and the peak inspiratory flow rates achieved for the inhaler device can be recorded. As mentioned above, the MEDSPRAY (MIST Beta) inhaler device was designed to provide a flow rate of about 15 L/min with moderate effort. In certain aspects, an inhaler device used in the methods described herein achieve a peak inhalation flow rate under moderate effort of at least about 15 L/min. In other embodiments, the inhaler device used in the methods described herein achieve a peak inhalation flow rate under moderate effort of greater than about 15 L/min. In yet additional aspects, the inhaler device used in the methods described herein achieve a peak inhalation flow rate under moderate effort of at least about 20 L/min, at least about 25 L/min, at least about 30 L/min, at least about 35 L/min, or at least about 40 L per min. In yet further aspects, the inhaler device used in the methods described herein achieve a peak inhalation flow rate under moderate effort of between about 15 and about 40 L/min, between about 20 and about 40 L/min, or between about 30 and 40 L/min.

As discussed above, SMI devices enable the use of propellant-free drug solutions. However, it is envisioned that the inclusion of a propellant in the formulation can, under certain circumstances, be advantageous. For example, the inclusion of a propellant may result in reduced aerosol droplet size due to evaporation of the propellant. Therefore, in some embodiments, the formulation can include a propellant.

In yet additional embodiments, the formulation described herein is propellant-free.

The aim of pulmonary administration is to deliver aerosol particles comprising the active ingredient to the lungs. Higher lung deposition translates to higher systemic absorption. In addition, because aerosols with high lung deposition exhibit less deposition in the throat and/or upper airways, this results in less coughing and irritation. Particles that are 5 microns or smaller are most likely to deposited in the lungs. The proportion of particles in an aerosol that are less than 5 microns in size is referred to as the fine particle fraction (FPF) whereas the term fine particle dose (FPD) is the absolute mass of drug particles that are less than 5 microns. Aerosols with higher FPFs have higher likelihood of depositing in the lungs and are associated with reduced irritation of the respiratory tract as opposed to the upper airway. The FPF of the aerosol produced by the RESPIMAT inhaler can be at least twice that of most pMDIs and DPIs. In addition, the FPF is higher for ethanol-based formulations as compared to aqueous formulations (Anderson 2006). Indeed, the higher FPF of an SMI aerosol translates in a higher proportion of the emitted dose delivered to the lungs as opposed to the oropharynx (Anderson 2006).

The present invention is directed to improved methods and compositions for pulmonary administration of cannabinoids. In addition to the high systemic absorption, pulmonary administration has a further advantage of avoiding hepatic first-pass metabolism. Advantages of the pulmonary administration also include a faster onset of action (the time it takes an active ingredient to reach a minimum effective concentration after the active ingredient is administered), greater stability, greater bioavailability, and/or or reduced individual variability of bioavailability, or, in the case of THC, a more intense psychotropic effect as compared to oral formulations such as MARINOL® and may be formulated for immediate release.

As discussed above, pulmonary administration of cannabinoids using inhaler devices has been limited by the irritation that orally inhaled cannabinoids induce in the throat and respiratory tract. Cannabinoids, such as Δ9-THC, Δ8-THC, and CBD, induce irritation at mucosal membranes and this irritation is dose-dependent. The aerosol formulation described herein is delivered using a soft mist inhaler. Cannabinoids as well as terpenes have good solubility in ethanol and thus a formulation for the soft mist inhaler can comprise a cannabinoid in a solution comprising ethanol. However, ethanol itself can irritate the mucosal membranes of the respiratory tract in a concentration and dose-dependent manner. In addition, evaporation of ethanol may result in aerosol particle size reduction and concentration of the cannabinoid in the droplet which may further exacerbate the throat and respiratory tract irritation experienced by the user.

The present invention is at least partially based on the discovery that saccharin or a salt thereof, such as sodium saccharin, can be added to a formulation comprising a cannabinoid in an ethanol solution and that the inclusion of the saccharin or the salt thereof results in less cough and respiratory tract irritation than that observed using the ethanol solution in the absence of the saccharin or salt thereof. As discussed above, the invention encompasses a method for pulmonary administration of an aerosol formulation comprising a cannabinoid, wherein the method comprises administering the formulation to a subject in an aerosol formulation by oral inhalation, wherein the aerosol formulation comprises the cannabinoid in a solution comprising ethanol, wherein the formulation further comprises saccharin or a salt thereof; and wherein the administration of the aerosol formulation induces less coughing, less throat irritation, and/or less respiratory tract irritation than the administration of an identical formulation in the absence of the saccharin or the salt thereof. The invention also includes a method of reducing cough and/or throat and/or respiratory tract irritation) caused by pulmonary administration of a cannabinoid administration (e.g., cannabinoid-related irritation) comprising adding an irritation reducing amount of saccharin or a salt thereof to an aerosol formulation comprising ethanol and cannabinoid and loading or filling a soft mist inhaler with the aerosol formulation.

While saccharin-containing aerosol formulations reduced coughing and throat irritation, many users experienced chest discomfort or chest tightness after inhalation that lasted approximately 30 minutes or longer. Saccharin-containing formulations utilizing the acidic form of saccharin (pKa ˜1.9) had a pH of about 3. It has been discovered that an aerosol formulation that has a pH closer to neutral is better tolerated. Specifically, administration of a formulation having a pH of about 6.5 and containing sodium saccharin in place of saccharin (free acid) resulted in less chest discomfort or tightness as compared to the saccharin (free acid)-containing formulation.

As described above, the invention includes an aerosol formulation comprising saccharin or preferably, a salt of saccharin, that has a pH close to neutral. In some embodiments, the pH of the formulation is between about 4 and 7.5, or between about 5 and 7.5, or between about 6 and 7.5. Salts of saccharin include, for example, the sodium, potassium, calcium, and ammonium salts of saccharin. In certain aspects, the saccharin salt is sodium saccharin or calcium saccharin. In yet additional aspects, the saccharin salt used in the formulation is sodium saccharin.

The saccharin or salt thereof, such as sodium saccharin, can be included in the formulation in an amount effective to reduce cough and/or irritation (or an irritation reducing amount) as compared to the identical formulation in the absence of the saccharin or salt thereof. In certain aspects, the amount of the saccharin or salt thereof is about 2% w/w or less, or about 0.2% w/w/or less, or is between about 0.08% to about 0.2% w/w, or is about 1% w/w. In yet further aspects, the formulation comprises sodium saccharin in an amount between about 0.08 and 0.2% w/w; for example, about 0.1% w/w.

As discussed above, aerosols with higher FPFs are more likely to deposit in the lungs. In certain aspects, the FPF of the emitted dose of the formulation comprising saccharin or a salt thereof is at least about 70%, or at least about 80%. In yet, further aspects, the particles in the fine particle fraction have an aerodynamic diameter between 1 and 5 microns.

The ethanol concentration in the formulation described herein comprising saccharin or a salt thereof, is at least an amount that is sufficient to solubilize the cannabinoid in the formulation. In some embodiments, the ethanol in the formulation is at least 140 proof, or at least 180 proof. In yet additional embodiments, the ethanol is 200 proof. In yet additional aspects, the amount of ethanol is about 85% or less, about 82% or less, or about 80% or less. In additional aspects, the amount of ethanol is about amount of ethanol in the formulation is between about 60 and 97%, or between about 68 and about 97% w/w, or between about 60 and 85% w/w. In yet further aspects, the ethanol solution comprises water in an amount of about 40% (w/w) or less, or about 30% (w/w) or less, or about 20% (w/w) or less. In yet additional aspects, the ethanol solution comprises water in an amount between about 0 and about 30% or between about 0 and about 25% w/w. In certain aspects, the formulation comprises Δ9-THC and the solution comprises 30% w/w or less water. In other embodiments, the formulation comprises CBD and the solution comprises water in an amount of 40% w/w or less. In additional aspects, the formulation comprises Δ9-THC and CBD and the ethanol solution comprises water in an amount of 30% w/w or less. In additional aspects, the formulation comprises Δ9-THC or CBD, or a combination thereof, and the solution comprises 20% w/w. water. In yet further aspects, the ethanol solution comprises saline; for example, the ethanol solution can comprise ethanol and saline and no water, or can comprise ethanol, saline and water.

In certain specific aspects, the formulation comprises Δ9-THC in an amount between about 2 and about 10% w/w, for example, about 8% w/w. In yet other aspects, the formulation comprises CBD in an amount between about 2 and about 10% w/w; for example, about 8% w/w. In yet additional aspects, the formulation comprises Δ9-THC in an amount of about 4% w/w and CBD in an amount of about 4% w/w.

The amount of Δ9-THC (e.g., a distillate) in the formulation comprising saccharin or salt thereof can be between about 2 and 10% w/w; for example, between about 2 and about 8% w/w, or between about 4 and about 8% w/w. In specific aspects, the amount of Δ9-THC in the formulation is about 8% w/w. In additional aspects, the Δ9-THC is in an ethanol solution comprising water in an amount of about 40% w/w or less, or about 30% w/w or less, or about 20% w/w or less. In a further specific example, the formulation comprise Δ9-THC in an amount between about 2 and about 8% w/w in an ethanol solution comprising water in an amount of about 20% w/w, and ethanol in an amount between about 60 and 70% w/w, and wherein the formulation further comprises saccharin or salt thereof in the formulation in an amount between about 0.08 and 0.2% w/w (for example, about 0.1% w/w). In yet further embodiments, the formulation comprise Δ9-THC in an amount between about 2 and about 8% w/w in an ethanol solution comprising water in an amount of about 0% w/w, and ethanol in an amount between about 80 and about 85% w/w, and wherein the formulation further comprises sodium saccharin in amount of 0.08 and about 0.2% w/w (for example, about 0.1% w/w). In certain embodiments, the formulation comprises Δ9-THC in an amount of about 8% w/w in an ethanol solution comprising water in an amount of about 0% w/w, and ethanol in an amount between about 80 and about 85% w/w, and wherein the formulation further comprises sodium saccharin in amount of about 0.08 and about 0.2% w/w (for example, about 0.1% w/w).

The amount of CBD (e.g., an isolate) in the formulation comprising saccharin or a salt thereof can be an amount between about 2 and about 10% w/w; for example, between about 2 and about 8% w/w or between about 4 and about 8% w/w. In specific aspects, the amount of CBD in the formulation is about 8% w/w. In a further specific example, the formulation comprises CBD in an amount between about 2 and about 8% w/w in an ethanol solution comprising water in an amount of about 20% w/w, and ethanol in an amount between about 60 and about 70% w/w, and wherein the formulation further comprises saccharin or salt thereof in the formulation is an amount between about 0.08 and about 0.2% w/w. In yet further embodiments, the formulation comprises CBD in an amount between about 2 and about 8% w/w in an ethanol solution comprising water in an amount of about 20% w/w, and ethanol in an amount between about 60 and about 70% w/w, and wherein the formulation further comprises sodium saccharin in amount of about 0.1% w/w). In yet further embodiments, the formulation comprises CBD in an amount between about 2 and about 8% w/w in an ethanol solution comprising water in an amount of about 0% w/w, and ethanol in an amount between about 80 and about 85% w/w, and wherein the formulation further comprises sodium saccharin in amount of 0.08 and about 0.2% w/w (for example, about 0.1% w/w). In additional embodiments, the formulation comprises CBD in an amount of about 8% w/w in an ethanol solution comprising water in an amount of about 0% w/w, and ethanol in an amount between about 80 and about 85% w/w, and wherein the formulation further comprises sodium saccharin in amount of about 0.08 and about 0.2% w/w (for example, about 0.1% w/w).

The present invention is also based on the discovery that including saline in an ethanol solution comprising the cannabinoid results in less coughing and/or throat and/or respiratory tract irritation than that observed using a pure ethanol solution (e.g., 200 proof ethanol; comprising no saline) and/or a solution comprising water instead of the saline. As discussed above, the invention encompasses a method for pulmonary administration of a cannabinoid aerosol formulation, wherein the method comprises administering the aerosol formulation to a subject by oral inhalation using a soft mist inhaler, wherein the aerosol formulation comprises the cannabinoid in a solution comprising ethanol and saline, wherein the administration of the aerosol formulation induces less coughing, less throat irritation, and/or less respiratory tract irritation than the administration of a control formulation, and/or wherein the emitted dose of the aerosol formulation is greater than that of a control formulation. The control formulation in this context is a formulation identical to the cannabinoid aerosol formulation except that the solution comprises no saline or comprises pure water instead of or in place of the saline. The invention also includes a method for reducing cannabinoid-related irritation and/or coughing of an aerosol formulation comprising a cannabinoid, the method comprising preparing an aerosol formulation comprising an irritation-reducing amount of saline in ethanol and loading a soft mist inhaler with the aerosol formulation, wherein the formulation comprises a cannabinoid. The invention additionally encompasses a method for increasing the emitted dose of the aerosol formulation using a soft mist inhaler as compared to that of a control formulation, the method comprising preparing an aerosol formulation comprising saline in ethanol, wherein the formulation comprises a cannabinoid, and loading a soft mist inhaler with the aerosol formulation, wherein the saline is present in an amount that increases the emitted dose.

Saline can be included in the formulations described herein in an amount effective to reduce cough and/or irritation (or an irritation reducing amount) and/or in an amount sufficient to increase the emitted dose as compared to the control formulation. The ethanol solution can, for example, comprise about 1% to about 30% by weight saline, about 1% to about 20% by weight saline, about 1% to about 10% by weight saline, or about 5 to about 10% by weight saline. In certain aspects, the amount of saline is between about 5 to about 10% by weight. In additional embodiments, the saline is present in the ethanol solution in an amount of about 5% by weight. In yet further aspects, the saline is present in the ethanol solution in amount of about 10% by weight.

The ethanol concentration in the formulation described herein comprising saline is at least an amount that is sufficient to solubilize the cannabinoid in the formulation. The amount of ethanol can, for example, be about 85% (w/w) or less, about 82% (w/w) or less, about 80% (w/w) or less. In additional aspects, the amount of ethanol is about amount of ethanol in the formulation is between about 60 and about 97%, or between about 68 and about 97% w/w, or between about 60 and 85% w/w. In certain aspects, the formulation comprises Δ9-THC and the ethanol solution comprises 30% w/w or less saline. In other embodiments, the formulation comprises CBD and the ethanol solution comprises saline in an amount of 40% w/w or less. In additional aspects, the formulation comprises Δ9-THC and CBD and the ethanol solution comprises saline in an amount of 30% w/w or less. In yet additional aspects, the formulation comprises Δ9-THC or CBD, or a combination thereof, wherein the ethanol solution comprises saline in an amount of about 20% w/w or less. In certain aspects, the formulation comprises Δ9-THC or CBD, or a combination thereof, wherein the ethanol solution comprises saline in an amount of about 10% w/w. The formulations described comprising saline can further comprise water. In certain aspects, the ethanol solution comprises water in an amount between about 0 and about 30% or between about 0 and about 25% w/w. For example, in some embodiments, the formulation comprises Δ9-THC or CBD, or a combination thereof, wherein the ethanol solution comprises saline in an amount of about 10% w/w, water in an amount of about 20% w/w and ethanol in amount between about 60 and 65% w/w. In yet other examples, the formulation comprises Δ9-THC or CBD, or a combination thereof, wherein the ethanol solution comprises saline in an amount of about 10% w/w, water in an amount of about 0% w/w and ethanol in amount between about 80 and about 85% w/w.

In certain specific aspects, the formulation comprises Δ9-THC in an amount between about 2 and about 10% w/w, for example, about 8% w/w. In yet other aspects, the formulation comprises CBD in an amount between about 2 and about 10% w/w; for example, about 8% w/w. In yet additional aspects, the formulation comprises Δ9-THC in an amount of about 4% w/w and CBD in an amount of about 4% w/w.

In some examples, the formulation can comprise Δ9-THC in an amount between about 2 and about 10% w/w in an ethanol solution comprising saline in an amount of about 10% w/w, water in an amount of about 20% w/w and ethanol in amount between about 60 and about 65% w/w. In another example, the formulation can comprise Δ9-THC in an amount between about 2 and about 10% w/w in an ethanol solution comprising saline in an amount of about 10% w/w, water in an amount of about 0% w/w and ethanol in amount between about 80 and about 85% w/w. In a further example, the formulation can comprise Δ9-THC in an amount of about 8% w/w in an ethanol solution comprising saline in an amount of about 10% w/w, water in an amount of about 20% w/w and ethanol in amount between about 60 and about 65% w/w. In another example, the formulation can comprise Δ9-THC in an amount of about 8% w/w in an ethanol solution comprising saline in an amount of about 10% w/w, water in an amount of about 0% w/w and ethanol in amount between about 80 and about 85% w/w.

In further examples, the formulation can comprise CBD in an amount between about 2 and about 10% w/w in an ethanol solution comprising saline in an amount of about 10% w/w, water in an amount of about 20% w/w and ethanol in amount between about 60 and about 65% w/w. In another example, the formulation can comprise CBD in an amount between about 2 and about 10% w/w in an ethanol solution comprising saline in an amount of about 10% w/w, water in an amount of about 0% w/w and ethanol in amount between about 80 and about 85% w/w. In a further example, the formulation can comprise CBD in an amount of about 8% w/w in an ethanol solution comprising saline in an amount of about 10% w/w, water in an amount of about 20% w/w and ethanol in amount between about 60 and about 65% w/w. In another example, the formulation can comprise CBD in an amount of about 8% w/w in an ethanol solution comprising saline in an amount of about 10% w/w, water in an amount of about 0% w/w and ethanol in amount between about 80 and about 85% w/w.

Osmolarity is the concentration of solution expressed as the total number of solutes (or osmoles) per liter. Osmolality is the number of osmoles in a kilogram of solvent. The normal human plasma osmolality is 275 to 299 mOsm/kg. In certain aspects, the osmolarity of the aerosol formulation is between about 15 to about 562 mOsm.

Irritation of airways and coughing can, for example, be measured by visual analog scales (VAS) with 0 cm (0%) on the 10-cm VAS scale standing for “not at all” and 10 cm (100%) for “very strong” as described in Meyer et al. (2018), Human Pharmacokinetics and Adverse Effects of Pulmonary and Intravenous THC-CBD Formulations. Med Cannabis Cannabinoids 1:36-43, the contents of which are expressly incorporated by reference herein.

As discussed above, aerosols with higher FPFs are more likely to deposit in the lungs. In certain aspects, the FPF of the emitted dose of the formulation (for example, comprising a cannabinoid in an ethanol solution comprising saline) is at least about 80%, at least about 82%, at least about 85%, at least about 87%, or at least about 90%. In yet, further aspects, the particles in the fine particle fraction have an aerodynamic diameter between 1 and 5 microns. In further aspects, the aerosol particles have a D90, based on particle volume basis, of less than about 8, less than about 7, or less than about 6.

Aerodynamic diameter can be determined by impactor studies such as those using multi-stage impactors and fast screening impactors. Using multi-stage cascade impactors, such as the Anderson cascade impactor and next generation impactor, the diameter is commonly reported as the mass median aerodynamic diameter (MMAD) and geometric Standard Deviation (GSD) which describe the particle size and size distribution, respectively. Multi-stage impactors commonly have 8 stages which allow fractionation of the aerosol based on particle size and subsequent determination of MMAD. In addition, an abbreviated impactor set-up, Copley Scientific's ‘Fast Screening Impactor’ (FSI), can be used to determine the FPF within liquid aerosols (Mitchell et al. AAPS PharmSciTech. 2009;10(1):252-7). The FSI consists of the universal induction port (UIP), course fraction collector (pre-separator and insert with a 5μm diameter cut-off at a specified flow rate, e.g. 28.3 L/min), and fine particle fraction collector consisting of a holder and glass fiber filter. The system can be assembled along with a Copley LC5 pump. The flow rate is set by attaching a flow meter to the inlet of the induction port and adjusting the flow control valve on the LC5 vacuum pump until the desired flow rate is achieved (e.g., 28.3 L/min +/−5%). Measurements are performed under ambient laboratory conditions (˜60% RH, 72° F.). For example, the SMI or Respimat device is primed and attached to the UIP using a mouthpiece adapter. With the vacuum pump running, the device is actuated followed by a 30 second hold before turning off the vacuum pump. The SMI or Respimat device is re-primed and another dose was collected in the FSI. To determine the amount of cannabinoids deposited in each stage, the FSI is dismantled and each stage is extracted with 10 ml of methanol. Samples are diluted and HPLC performed to quantify the mass of cannabinoids deposited in the USP throat, coarse fraction collector, and fine particle fraction collector.

The aerosol particle size is related to the viscosity of the solution, the surface tension of the solution, and the volatility of the solvent (for example, the volatility of ethanol). In some embodiments, the viscosity of the formulation described herein is 2 cP or less.

In certain embodiments, the formulation of the present invention has a Tmax that is about 3 to about 15 minutes. In some embodiments, the composition of the present invention has a Tmax that is about 1 to about 10 minutes. In a further embodiment, the Tmax is about 10 to about 60 minutes. In a further embodiment, the Tmax is about 1 to about 2 hours.

In additional aspects, the maximum amount of the cannabinoid in the formulation comprising saccharin or a salt thereof that can be administered without inducing moderate or severe coughing and/or throat and/or respiratory tract irritation is at least about 1.2, about 1.5 times, or 2.0 times more than the maximum amount of the cannabinoid that can be dosed in the formulation without saccharin or the salt thereof without causing moderate or severe coughing and/or throat and/or respiratory tract irritation.

In additional aspects, the maximum amount of the cannabinoid in the formulation comprising saline described herein that can be administered without inducing moderate or severe coughing and/or throat and/or respiratory tract irritation is at least about 1.2, about 1.5 times, or about 2.0 times more than the maximum amount of the cannabinoid that can be dosed in a control formulation without causing moderate or severe coughing and/or throat and/or respiratory tract irritation.

### Cannabinoids

The chemical structures of Δ9-THC, Δ8-THC, cannabidiol (CBD), cannabinol (CBN), cannabidivarin (CBDV), cannabigerol (CBG), tetrahydrocannabivarin (THCV), cannabicyclol (CBL), cannabichromene (CBC), cannabivarin (CBV), cannabigerovarin (CBGV), cannabichromevarin (CBCV), and cannabigerol monomethyl ether (CBGM), which can be used in the cannabinoid formulation, are shown below:

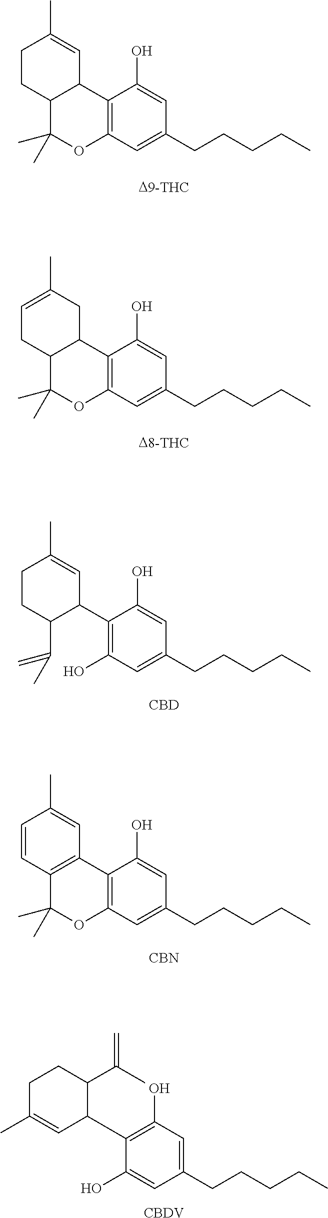

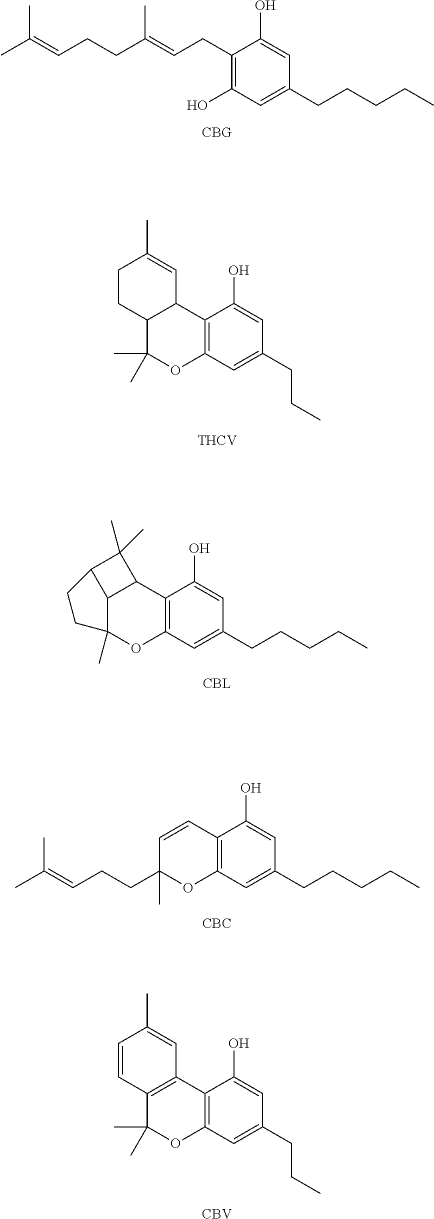

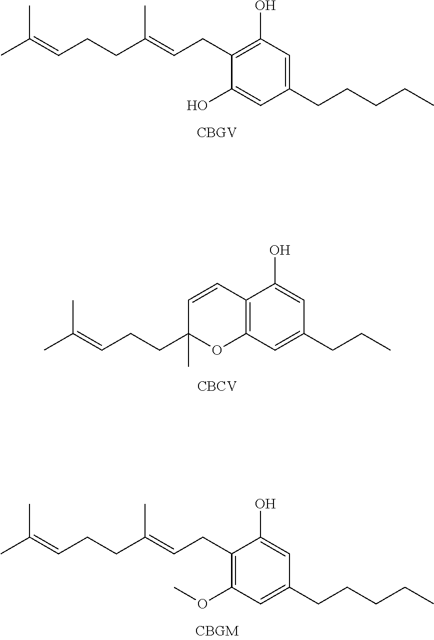

Certain cannabinoids, like Δ9-THC, can have three fused rings and these rings are referred to in the literature as the A-ring, B-ring and C-ring. For example, Formula (a) below shows the structure of THC, where the dashed line represents either a double bound between 8-9 (Δ8-THC) or between 9-10 ( Δ9-THC). As illustrated below, certain cannabinoids lack one or two of rings A, B, or C, e.g., CBC (Formula (b)), CBL (Formula (c)) or CBD (Formula (d).

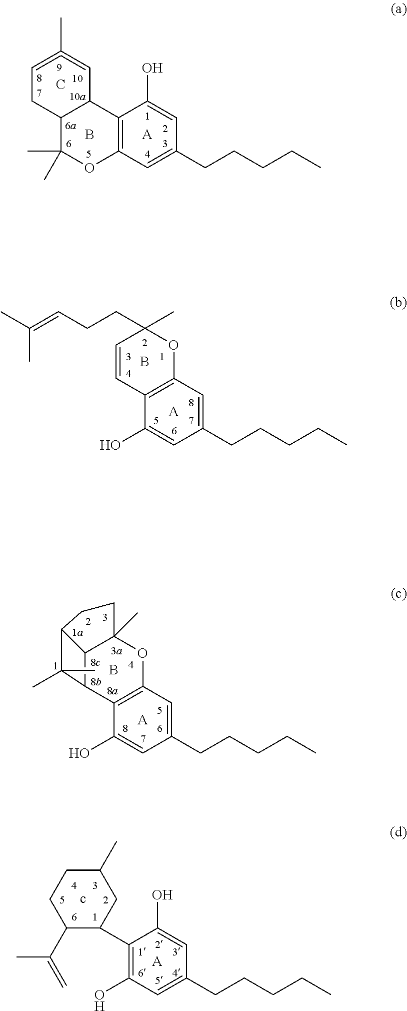

Cannabinoids that can be used in the methods and formulations of the present invention include, but are not limited, to: tetrahydrocannabinol (THC), Δ9-tetrahydrocannabinol (Δ9-THC), Δ8-tetrahydrocannabinol (Δ8-THC), tetrahydrocannabinolic acid (THCA), cannabinolic acid (CBNA), Δ8-tetrahydrocannabinol-dimethylheptyl, Δ9-tetrahydrocannabinol-dimethylheptyl, Δ9-tetrahydrocannabinol propyl analogue (THCV), 11-nor-9-carboxy-tetrahydrocannabinol, 5′-azido-Δ8-tetrahydrocannabinol, AMG-1, AMG-3, AM411, AM855, nabilone, HU-210, dexanabinol (HU-211), HU-308, O-1184, JWH-051, AM087, cannabivarin (CBV), tetrahydrocannabivarin (THCV), cannabichromevarin (CBCV), cannabigerol monomethyl ether (CBGM), cannabinol (CBN), cannabichromene (CBC), cannabichromene acid (CBCA) cannabichromene propyl analogue, cannabicyclol (CBL), levonantradol (CP 50556-1), 19,19-Dimethylheptyl-D-8-tetrahydrocannabinol-11-oic acid (CT-3), 9-carboxy-11-norcannabinol, 1′-oxocannabinol, 11-nor-Δ8-THC-9-carboxylic acid, 2′-carboxy-3′,4′,5′-trinor-Δ9-THC, 5′-carboxy-Δ9-THC, 9-carboxy-11-nor-Δ9-THC, 9-carboxy-11-nor-Δ8-THC, [(6aR,10aR)-3-[(1S,2R)-1,2-dimethylheptyl]-6a,7,10,10a-tetrahydro-6, 6,9-trimethyl-6H-dibenzo[b,d]pyran-1-ol], 9-carboxy-11-nor-(2 or 4)-chloro-Δ8-THC, 5′-Dimethylamino-Δ8-THC, 5′-methylamino-Δ8-THC, 5′-N-methyl-N-4-(7-nitrobenzofurazano)amino-Δ8-THC, (−)-trans-Δ8-THC, 5′-trimethylammonium-Δ8-THC phenolate, cannabidiolic acid (CBDA), cannabigerolic acid (CBGA), 11-hydroxy-tetrahydrocannabinol, AM938, AM708, AM836, CP 55940, CP 55244, AM919, AM926, dimethylheptyl HHC, cannabidiol (CBD), cannabidivarin (CBDV), cannabigerovarin (CBGV), cannabidiol propyl analogue (CBDV), cannabigerol (CBG), cannabielsoin (CBE), cannabinodiol (CBDL), IP-751 (ajulemic acid), desacetyl-L-nantradol, CP 47497, cannabicyclohexanol (CP-47,497 C8 homolog), CP 47497 C6 homolog, CP 47497 C7 homolog, or CP 47497 C9 homolog, 10-hydroxycannabidiol, 1′-hydroxycannabinol, 11-hydroxycannabinol, 11-hydroxy-Δ9-THC, 1′-hydroxy-Δ9-THC (Isomer B), 11-hydroxy-Δ8-THC, 2′-hydroxy-Δ9-THC, 3′-hydroxy-Δ9-THC, 4′-hydroxy-Δ9-THC, 5′-hydroxy-Δ9-THC, 8α-hydroxy-Δ9-THC, 8β-hydroxy-Δ9-THC, 5′-Trimethylammonium-11-hydroxy-Δ8-THC phenolate, cannabinodiol (CBND), cannabitriol (CBTL), 8α-11-dihydroxy-Δ9-THC, or 8β-11-Dihydroxy- Δ9-THC.

Additional cannabinoids have also been described, for example, in Thakur et al. (2005). Structural Requirements for Cannabinoid Receptor Probes, HEP 168: 209-246: Seltzman et al. (1999). Structure and receptor activity for Classical Cannabinoids, Current Medicinal Chemistry 6:685-704; Bow et al (2016). The Structure—Function Relationships of Classical Cannabinoids: CB1/CB2 Modulation. Perspectives in Medicinal Chemistry 2016:8 17-39 doi: 10.4137/PMC. S32171; the contents of each of which are expressly incorporated by reference herein.

In a preferred embodiment, the cannabinoid is selected from the group consisting of THC, THCA, THCV, CBD, CBDA, CBDV, CBDL, CBC, CBCA, CBCV, CBCN, CBV, CBG, CBGA, CBGV, CBN, CBL, and CBE, or a combination of any of thereof. In certain additional aspects, the cannabinoid is selected from the group consisting of CBG, THCV, CBN, THC, CBC, CBD, and CBDV. In another embodiment, the cannabinoid is selected from the group consisting of THC, CBD, THCA, and CBDA, or a combination of any of thereof. In another embodiment, the cannabinoid is THC or CBD, or a combination thereof. In another embodiment, the THC is Δ9-THC or Δ8-THC, or a combination thereof. In another embodiment, the THC is Δ9-THC. In a further specific aspect, the cannabinoid is CBD. In yet an additional aspect, the formulation comprises a combination of Δ9-THC and CBD.

In additional aspects, the cannabinoid is a naturally occurring cannabinoid. Naturally occurring cannabinoids include cannabinoids that can be extracted from or isolated from the Cannabis sativa, Cannabis indica, or cannabis hybrid plants.

The formulations described herein can further comprise a terpene.

Cannabinoids and terpenes can be purchased or synthesized using well-known techniques. Cannabinoids can be extracted from a plant using well-known methods. Specifically, cannabinoids and terpenes can be extracted from a plant of the Cannabis genus, e.g., Cannabis sativa, Cannabis indica, or Cannabis hybrid. Terpenes can also be extracted from a plant that is not a member of the Cannabis genus. Phytocannabinoids and terpenes may be extracted as terpene blends or, in the case of a Cannabis species, as a cannabinoid or cannabinoid/terpene blend. The blends may be used directly or can be separated into individual or fewer components using distillation (e.g., short-path rotary distillation) or other techniques. The relative amount of each principal phytocannabinoid and/or terpene in the plant extract, e.g., cannabis extract, varies according to the cannabinoid and/or terpene profile and levels of the particular plants and methodology of extraction. Extracts comprising terpenes, e.g., extracts essentially free of cannabinoids, extracts that contain cannabinoids as a minor constituent, or extracts from a plant that is not a species of Cannabis (e.g., Cannabis sativa, Cannabis indica, Cannabis hybrid, or other), i.e., a non-Cannabis species, may be used individually or combined with one or more other active ingredients, e.g., cannabinoids or cannabinoid extracts.

Cannabinoids and/or terpenes can be obtained by separating resins from leaves or leaves and flowers of cannabis plants by solvent extraction. Extracts derived from cannabis plants include primary extracts prepared by such processes as, for example, maceration, percolation, and solvent extraction. Solvent extraction can be carried out using a solvent that dissolves cannabinoids/cannabinoid acids, such as for example C1 to C5 alcohols (e.g. ethanol, methanol), C3-C12 alkanes (e.g. hexane, butane or propane), Norflurane (HFA134a), HFA227, and carbon dioxide. General protocols for the preparation of extracts of cannabis plant material are described in U.S. Pat. App. Pub. No 20060167283 (WO 02/064109), which is incorporated herein by reference. Carbon dioxide provides another method to extract cannabinoid/terpene resins from cannabis plant material. Sub Critical (Liquid) or Supercritical CO₂ is forced through the plant matter, which separates the cannabinoid/terpenes from the plant matter resulting in a transparent, amber oil. Primary extracts obtained by supercritical fluid extraction (SFE) may undergo an ethanolic precipitation step in order to remove less polar, plant derived impurities (e.g., lipids). The extracts obtained by supercritical fluid extraction (SFE) may undergo a secondary extraction, e.g. an ethanolic precipitation, to remove non-cannabinoid/terpene materials. In a preferred embodiment, light petroleum gas extraction, using a LHBES (light hydrocarbon butane extraction system) 1300/C from ExtractionTek Solutions is used to extract cannabinoids from cannabis plant material.

A modified extraction process consists of decarboxylating the starting concentrate at 300° F. until fully converted and the bubbling stops. Once the oil is decarboxylated, it is run through the VTA-VKL 70-5 short path rotary distillation plant twice. The first run separates the heavy terpenes and lighter terpenes from the cannabinoids and waste material. The cannabinoids and waste are run through again with a higher vacuum and higher temperature to separate the cannabinoids from the remaining waste. The waste is collected and run again in a larger batch to extract all cannabinoids and terpenes. The VTA-VKL 70-5 short path rotary distillation plant uses a top stirring rotary column to wipe incoming product into a thin film for better heat distribution and evaporation. The inner condensing column is set to condense the cannabinoids into liquids. The waste and cannabinoids are diverted into the two dispensing arms for collection into receiving vessels. The light terpenes are collected in a receiving flask attached to the inline chiller on the plant. The system (except for feed vessel) are under vacuum during the operation. The vacuum for the first run should be between 0.5-0.7 mbar. For the second run, pressure should be between 0.5-0.07 mbar.

In one embodiment, the cannabinoid is in the form of an extract from a cannabis plant comprising a cannabinoid, i.e., a “cannabinoid extract”. The formulation can further comprise a terpene. In some embodiments, the terpene is in the form of an extract from cannabis or other plant comprising a terpene, i.e., a “terpene extract” In a further embodiment, the cannabinoid or terpene extract is from a cannabis plant selected from Cannabis sativa, Cannabis indica, or Cannabis hybrid. In one embodiment, the cannabinoid or terpene extract is an extract of Cannabis sativa. In another embodiment, the cannabinoid or terpene extract is an extract of Cannabis indica. In another embodiment, the cannabinoid or terpene extract is an extract of Cannabis hybrid. In another embodiment, the cannabinoid or terpene extract is a distillate. In a further embodiment, the cannabinoid distillate is the product of short path distillation of a cannabinoid extract. In a further embodiment, the cannabinoid or terpene is synthetic.

In further embodiments, the cannabinoid extract comprises total cannabinoid(s) in an amount selected from: 50-75 wt %, 50-99 wt %, 75-99 wt %, 75-95 wt %, 80-99 wt %, 85-99 wt %, 90-99 wt %, 85-95 wt %, 90-95 wt %, or >99 wt % total cannabinoid(s). In further embodiments, the total concentration of cannabinoid(s) in a composition of the present invention is 1-200 mg/mL. In further embodiments, the total concentration of cannabinoid(s) in a composition of the present invention is selected from: 1-5 mg/mL, 1-10 mg/mL, 1-50 mg/mL, 1-100 mg/mL, 5-50 mg/mL, 10-50 mg/mL, 10-100 mg/mL, 5-10 mg/mL, 10-15 mg/mL, 15-20 mg/mL, 20-30 mg/mL, 30-40 mg/mL, 40-50 mg/mL, 50-75 mg/mL, 75-100 mg/mL, 100-150 mg/mL, or 150-200 mg/mL. In another embodiment, the total concentration of cannabinoid(s) in a composition of the present invention is <0.001 mg/mL, 0.001-0.01 mg/mL, or 0.01-1 mg/mL.

In one embodiment, the composition or formulation described herein comprises at least one terpene. In one embodiment, the terpene is found in Cannabis sativa, Cannabis indica, or Cannabis hybrid. In a further embodiment, the terpene is extracted from a plant species, preferably a species of Cannabis (e.g., Cannabis sativa, Cannabis indica, Cannabis hybrid or other). In a further embodiment, the terpene is synthetic. In a further embodiment, the terpene is selected from any, one, two, three, four, five, six, seven, eight, nine, ten, eleven, twelve, thirteen, fourteen, fifteen, sixteen, seventeen, eighteen, nineteen, twenty, or more of the group consisting of: alpha-bisabolol, alpha-phellandrene, alpha-pinene, alpha-terpinene, alpha-terpineol, beta-caryophyllene, α-pinene, beta-pinene, borneol, cadinene, camphene, camphor, carvacrol, β-caryophyllene, caryophyllene acetate, caryophyllene oxide, cedrane, citral, citronellol, dextro carvone, dextro fenchone, eucalyptol (1,8-cineole), eugenol, farnesene, gama-3-carene, gamma-terpinene, geraniol, geranyl acetate, guaiene, humulene, isopulegol, limonene, linalool, linalyl acetate, menthol, myrcene, β-myrcene, nerol, nerolidol, ocimene, ocimene, p-cymene, phytol, pulegone, terpineol, terpinen-4-ol, terpinolele, terpinolene, thymol, valencene, valencene,1-menthol, and combinations thereof.

As described above, the formulations described herein can comprise a terpene alone or in combination one or more cannabinoid. In a further embodiment, the at least one terpene is any one, two, three, four, five, six, or all six terpenes selected from the group consisting of beta-caryophyllene, linalool, limonene, alpha-pinene, eucalyptol, and myrcene. In a further embodiment, the at least one terpene is any one, two, three, four, or all five selected from beta-caryophyllene, linalool, limonene, alpha-pinene, or eucalyptol. In yet an additional aspect, the at least one terpene is any one, two, three, four, or all five selected from beta-caryophyllene, α-pinene, β-pinene, eucalyptol, and limonene. In certain embodiments, the at least one terpene is a blend comprising beta-caryophyllene, α-pinene, β-pinene, eucalyptol, and limonene. In yet further aspects, the blend comprises about 40% w/w beta-caryophyllene, about 15% w/w α-pinene, about 15% w/w β-pinene, about 10% w/w eucalyptol, and about 20% w/w limonene.

In some embodiments, the cannabinoid is Δ9-THC and the dose of the Δ9-THC administered to the subject is effective to induce a psychoactive effect. In additional aspects, the cannabinoid is Δ9-THC, and the amount of the cannabinoid is a therapeutically effective amount. In yet further aspects, the cannabinoid is CBD and the amount of the CBD is a therapeutically effective amount. In yet additional aspect, the formulation comprises a Δ9-THC and CBD, wherein the Δ9-THC and CBD are each present in an effective amount or a therapeutically effective amount.

The dose of cannabinoid emitted by the device is related to the concentration of the cannabinoid in the formulation (for example, % w/w) and the density of the solution. In certain aspects, the dose of the cannabinoid administered is greater than about 0.2 mg, greater than about 0.35 mg, greater than about 0.5 mg. The dose of cannabinoid can be administered by one puff or actuation of the device, or more than one puff or actuation. For example, the desired dose can be administered with a single actuation or a plurality of actuations.

In one embodiment, the composition, e.g., cannabinoid composition, of the present invention has an onset of action within 15 minutes, 15-20 minutes, 20 minutes, 25 minutes, 30 minutes, or within 45 minutes post administration.

In one embodiment, the composition, e.g., cannabinoid composition, of the present invention has a peak time within 90 minutes, within 80 minutes, within 70 minutes, within 60-70 minutes, within 60 minutes, within 50 minutes, within 45-60 minutes, within 45 minutes, within 40 minutes, or within 30 minutes post administration.

As described herein, when the coughing, throat irritation, and/or respiratory tract irritation of an SMI formulation comprising a cannabinoid in an ethanol solution that further comprises saccharin or a salt thereof is compared to an identical formulation in the absence of saccharin or a salt thereof, it is to be understood that all other conditions of the administration are identical (including, for example, the dose of the cannabinoid, the device, the inhalation technique, etc.).

Similarly, when the coughing, throat irritation, and/or respiratory tract irritation of an SMI formulation comprising a cannabinoid in a solution comprising ethanol and saline is compared to a control formulation, it is to be understood that all other conditions of the administration are identical (including, for example, the dose of the cannabinoid, the device, the inhalation technique, etc.).

In certain embodiments, the formulation or composition does not include any additional ingredients that would decrease coughing and/or irritation. In other aspects, the formulation or composition does not include any additional ingredients that would decrease the coughing and/or irritation in an amount sufficient to reduce said coughing and/or irritation. In certain aspects, the formulation or composition is free of an additional agent selected from a medium chain triglyceride, a propylene glycol diester, a human TAS2R bitter taste receptor agonist(s), a local anesthetic(s), and/or a cough suppressant. In certain additional aspects, the formulation or composition does not include an additional agent selected from a medium chain triglyceride, a propylene glycol diester, a human TAS2R bitter taste receptor agonist(s), a local anesthetic(s), and/or a cough suppressant in an amount sufficient to reduce said coughing and/or irritation. In yet other aspects, the formulation or composition includes an additional agent selected from a medium chain triglyceride, a propylene glycol diester, a human TAS2R bitter taste receptor agonist(s), a local anesthetic(s), and/or a cough suppressant but the formulation comprising saccharin or a salt thereof induces less coughing and/or less throat irritation and/or less upper or lower respiratory irritation than an identical composition in the absence of saccharin or a salt thereof. In further aspects, the formulation or composition includes a medium chain triglyceride, a propylene glycol diester, a human TAS2R bitter taste receptor agonist(s), a local anesthetic(s), and/or a cough suppressant but the formulation comprising saline induces less coughing and/or less throat irritation and/or less upper or lower respiratory irritation than a control formulation.

In yet additional aspects, the formulation comprising ethanol and saline as described herein comprises an additional ingredient that decreases coughing and/or irritation. In certain aspects, the formulation or composition comprises a medium chain triglyceride, a propylene glycol diester, a human TAS2R bitter taste receptor agonist(s), a local anesthetic(s), and/or a cough suppressant. In certain additional aspects, the formulation or composition comprises a medium chain triglyceride, a propylene glycol diester, a human TAS2R bitter taste receptor agonist(s), a local anesthetic(s), and/or a cough suppressant in an amount sufficient to reduce said coughing and/or irritation. In yet other aspects, the formulation or composition comprises a medium chain triglyceride, a propylene glycol diester, a human TAS2R bitter taste receptor agonist(s), a local anesthetic(s), and/or a cough suppressant but the formulation comprising saline induces less coughing and/or less throat irritation and/or less upper or lower respiratory irritation than a control formulation.

In certain specific embodiments, the formulation described herein does not include a local anesthetic. In other aspects, the formulation does not comprise a local anesthetic in an amount effective to decrease the coughing and/or irritation in an amount sufficient to reduce said coughing and/or irritation. For example, the formulation does not include a local anesthetic that is an aminoamide or an aminoester, for example, anesthetic selected from the group consisting of lidocaine, articaine, mepivicaine, etidocaine, prilocaine, bupivacaine, procaine, tetracaine, benzocaine and chloroprocaine (includes the free base of the local anaesthetic and pharmaceutical acceptable salts thereof).

### Soft Mist Inhaler Device Comprising the Formulation

The invention also includes a soft mist inhaler device comprising a reservoir or cannister or inhaler body for storing the cannabinoid formulation, and wherein said reservoir or cannister is at least partially filled with a formulation described herein.

In certain specific aspects, the inhaler is a portable soft mist inhaler for propellant-free metered nebulization of a cannabinoid formulation, comprising:

an inhaler body, and

a mechanical pressure generator for pressurizing a supply of the cannabinoid formulation located in the inhaler body,

an expulsion nozzle for receiving the formulation and for delivering the received dose of the nebulized medicament preparation as an aerosol;

wherein the cannabinoid formulation comprises a cannabinoid in a solution comprising ethanol, and wherein the formulation further comprises saccharin or a salt thereof,

wherein administration of the aerosol formulation induces less coughing, less throat irritation, and/or less respiratory tract irritation than the administration of an identical formulation in the absence of the saccharin or the salt thereof.

In certain additional embodiments, the inhaler is a portable soft mist inhaler for propellant-free metered nebulization of a cannabinoid formulation, comprising:

an inhaler body, and

a mechanical pressure generator for pressurizing a supply of the cannabinoid formulation located in the inhaler body,

an expulsion nozzle for receiving the formulation and for delivering the received dose of the nebulized medicament preparation as an aerosol;

wherein the aerosol formulation comprises the cannabinoid in a solution comprising ethanol and saline,

wherein the administration of the aerosol formulation induces less coughing, less throat irritation, and/or less respiratory tract irritation than the administration of a control formulation, and/or

wherein the emitted dose of the aerosol formulation is greater than that of a control formulation;

wherein the control formulation is a formulation identical to the cannabinoid aerosol formulation except that the solution comprises no saline or comprises pure water instead of the saline.

The cannabinoid-containing or terpene-containing formulation is stored as a solution in the drug cartridge or inhaler body which can, for example, be an aluminum cylinder containing a double-walled, plastic, collapsible bag that contracts as the solution is used. The number of actuations per device can be, for example, about 50, about 60 about 100, about 120, or more than about 100.

### Additional Actives

In one embodiment, the formulation described herein comprises at least two active ingredients, wherein at least one of the active ingredients is the cannabinoid. In addition to the cannabinoid, the composition may contain, e.g., one or more additional cannabinoids, terpenes, or other additional non-cannabinoid active ingredient(s). In one embodiment, at least one of the other additional active ingredients, i.e., in addition to the cannabinoid, is selected from one or more cannabinoid, cannabinoid extract, terpene, terpene extract, anti-insomnia, anti-tussive, opioid analgesic, decongestant, non-opioid analgesic/anti-inflammatory drug, anti-migraine drug, anti-emetic, anti-histamine, proton pump inhibitor, H₂ antagonist/H₂ blocker, tranquilizer, anticonvulsant, hypnotic, muscle relaxant, anti-psychotic, anti-diarrheal, Attention Deficit and Hyperactivity Disorder (ADHD) drug, anti-Parkinson disease drug, benzodiazepine, benzodiazepine antagonist, barbiturate, barbiturate antagonist, stimulant, stimulant antagonist, antidepressant, nutraceutical, nicotine, BCS Class II active ingredient, BCS Class IV active ingredient, or a combination thereof.

In one embodiment, the formulation described herein comprises at least two active ingredients, wherein at least one of the active ingredients is a terpene. In addition to the terpene, the composition may contain, e.g., one or more additional cannabinoids, terpenes, or other additional non-cannabinoid active ingredient(s). In one embodiment, at least one of the other additional active ingredients, i.e., in addition to the terpene, is selected from one or more cannabinoid, cannabinoid extract, terpene, terpene extract, anti-insomnia, anti-tussive, opioid analgesic, decongestant, non-opioid analgesic/anti-inflammatory drug, anti-migraine drug, anti-emetic, anti-histamine, proton pump inhibitor, H₂ antagonist/H₂ blocker, tranquilizer, anticonvulsant, hypnotic, muscle relaxant, anti-psychotic, anti-diarrheal, Attention Deficit and Hyperactivity Disorder (ADHD) drug, anti-Parkinson disease drug, benzodiazepine, benzodiazepine antagonist, barbiturate, barbiturate antagonist, stimulant, stimulant antagonist, antidepressant, nutraceutical, nicotine, BCS Class II active ingredient, BCS Class IV active ingredient, or a combination thereof.

In another embodiment, the additional active ingredient(s) comprises an anti-insomnia drug. In further embodiments, the anti-insomnia drug is selected from any one or more of: melatonin, trazodone, zolpidem, temazepam, elprazolam, amitriptyline, halcion, lorazepam, clonazepam, Intermezzo, eszopiclone, diphenhydramine, doxepin, mirtazapine, gabapentin, doxylamine, quetiapine, flurazepam, estazolam, olanzapine, Seconal, triazolam, zaleplon, secobarbital, chloral hydrate, oxazepam, quazepam, ramelteon, suvorexant, butabarbital, pentobarbital, phenobarbital, phenyltoloxamine, amobarbital, diphenhydramine, dimenhydrinate, diphenhydramine/magnesium salicylate, diphenhydramine/naproxen, diphenhydramine/aspirin, diphenhydramine/paracetamol, diphenhydramine/ibuprofen, or tasimelteon.

In some embodiments, the additional active ingredient(s) comprise an anti-tussive. In further embodiments, the anti-tussive is selected from any one or more of: benzonatate, caramiphen edisylate, chlophedianol, codeine, dextromethorphan hydrobromide, hydrocodone, levopropoxyphene, morphine, codeine, ethylmorphine, dihydrocodeine, benzylmorphine, laudanum, dihydroisocodeine, nicocodeine, nicodicodeine, hydrocodone, hydromorphone, acetyldihydrocodeine, thebacon, diamorphine (heroin), acetylmorphone, noscapine, or pholcodine.

In additional embodiments, the additional active ingredient(s) comprise an opioid analgesic. In further embodiments, the opioid analgesic is selected from any one or more of:

alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, or tramadol.

In additional embodiments, the additional active ingredient(s) comprise a decongestant. In further embodiments, the decongestant is selected from any one or more of: pseudoephedrine hydrochloride, phenylephrine bitartrate, phenylephrine hydrochloride or pseudoephedrine sulfate.

In certain additional embodiments, the additional active ingredient(s) comprise a non-opioid analgesic/anti-inflammatory drug. In further embodiments, the non-opioid analgesic/anti-inflammatory is selected from any one or more of: acetaminophen or a non-steroidal anti-inflammatory agent selected from the group consisting of aspirin, celecoxib, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, or isoxicam.

In additional aspects, the additional active ingredient(s) comprise an anti-migraine drug. In further embodiments, the anti-migraine drug is selected from any one or more of: 2-bromo-LSD, acetaminophen/dichloralphenazone/isometheptene mucate, almotriptin, alniditan, amidrine, avitriptan, caffeine/ergotamine, calcitonin gene-related peptide receptor antagonist, clonidine, dasolampanel, dihydroergotamine, dimetotiazine, donitriptan, dotarizine, eletriptan, ergotamine, ergotamine/chlorcyclizine/caffeine, flumedroxone acetate, iprazochrome, lasmiditan, lisuride, lomerizine, methysergide, migraleve, naratriptan, naproxen, naproxen/sumatripta, olcegepant, oxetorone, paracetamol/metoclopramide, prochlorperazine, promethazine, proxibarbital, rimegepant, rizatriptan, selurampanel, sumatriptan, telcagepant, tezampanel, topiramate, or zolmitriptan.

In one embodiment, the additional active ingredient(s) comprise an anti-emetic. In further embodiments, the anti-emetic is selected from any one or more of: dolasetron, granisetron, ondansetron, tropisetron, palonosetron, mirtazapine, metoclopramide, cyclizine, diphenhydramine, dimenhydrinate, meclizine, promethazine, or hydroxyzine.

In additional embodiments, the additional active ingredient(s) comprise an anti-histamine. In further embodiments, the anti-histamine is selected from any one or more of: diphenhydramine, loratadine, desloratadine, meclizine, fexofenadine, pheniramine, cetirizine, promethazine, brompheniramine, clemastine fumarate or chlorpheniramine.

In some embodiments, the additional active ingredient(s) comprise a proton pump inhibitors (PPI). In further embodiments, the PPI is selected from any one or more of: omeprazole, esomeprazole, pantoprazole, lansoprazole, or rabeprazole.

In some embodiments, the additional active ingredient(s) comprise a H₂ antagonist/H₂ blocker. In further embodiments, the H₂ antagonist/H₂ blocker is selected from any one or more of: cimetidine, ranitidine, or famotidine.

In some embodiments, the additional active ingredient(s) comprise a tranquilizer. In further embodiments, the tranquilizer is selected from any one or more of: amobarbital, pentobarbital, secobarbital, phenobarbital, clonazepam, diazepam, estazolam, flunitrazepam, lorazepam, midazolam, nitrazepam, oxazepam, triazolam, temazepam, chlordiazepoxide, or alprazolam.

In some embodiments, the additional active ingredient(s) comprise an anticonvulsant. In further embodiments, the anti-convulsant is selected from any one or more of: elbamate, carbamazepine, oxcarbazepine, vigabatrin, progabide, tiagabine, topiramate, gabapentin, pregabalin, ethotoin, phenytoin, valproic acid, or lamotrigine.

In some embodiments, the additional active ingredient(s) comprise a hypnotic. In further embodiments, the hypnotic is selected from any one or more of: zolpidem, zaleplon, zopiclone, or eszopiclone.

In some embodiments, the additional active ingredient(s) comprise a muscle relaxant. In further embodiments, the muscle relaxant is selected from any one or more of: methocarbamol, carisoprodol, chlorzoxazone, cyclobenzaprine, gabapentin, metaxalone, or orphenadrine.

In some embodiments, the additional active ingredient(s) comprise an anti-psychotic. In further embodiments, the anti-psychotic is selected from any one or more of: haloperidol, droperidol, chlorpromazine, fluphenazine, perphenazine, prochlorperazine, thioridazine, trifluoperazine, mesoridazine, promazine, triflupromazine, levomepromazine, methotrimeprazine, pimozide, chlorprothixene, flupenthixol, thiothixene, zuclopenthixol, clozapine, olanzapine, risperidone, quetiapine, ziprasidone, amisulpride, asenapine, or paliperidone.

In some embodiments, the additional active ingredient(s) comprise an anti-diarrheal. In further embodiments, the anti-diarrheal is bismuth subsalicylate or loperamide.

In some embodiments, the additional active ingredient(s) comprise an Attention Deficit and Hyperactivity Disorder (ADHD) drug. In further embodiments, the ADHD drug is selected from any one or more of: methylphenidate, dextroamphetamine sulfate, amphetamine, or atomoxetine hydrochloride.

In some embodiments, the additional active ingredient(s) comprise an anti-Parkinsons disease drug. In further embodiments, the anti-Parkinson disease drug is selected from any one or more of: amantadine, Apokyn, apomorphine, bromocriptine, carbidopa/levodopa, Cycloset, Duopa, entacapone/levodopa/carbidopa, Gocovri, levodopa, Mirapex, Mirapex ER, Neupro, Parlodel, pramipexole, Requip, Requip XL, ropinirole, rotigotine, Rytary, Sinemet, Sinemet CR, or Stalevo.

In some embodiments, the additional active ingredient(s) comprise a benzodiazepine. In further embodiments, the benzodiazepine is selected from any one or more of: alprazolam, bromazepam, chlordiazepoxide, clorazepate, diazepam, estazolam, flurazepam, halazepam, ketazolam, lorazepam, nitrazepam, oxazepam, prazepam, quazepam, temazepam, and triazolam. In some embodiments, the additional active ingredient(s) comprise is a benzodiazepine antagonist. In further embodiments, the benzodiazepine antagonist is flumazenil.

In some embodiments, the additional active ingredient(s) comprise a barbiturate. In further embodiments, the barbiturate is selected from any one or more of: amobarbital, aprobarbotal, butabarbital, butalbital, methohexital, mephobarbital, metharbital, pentobarbital, phenobarbital, and secobarbital.

In some embodiments, the additional active ingredient(s) comprise a barbiturate antagonist. In further embodiments, the barbiturate antagonist is an amphetamine.

In some embodiments, the additional active ingredient(s) comprise a stimulant. In further embodiments, the stimulant is selected from caffeine or an amphetamine, such as amphetamine, dextroamphetamine resin complex, dextroamphetamine, methamphetamine, or methylphenidate.

In some embodiments, the additional active ingredient(s) comprise a stimulant antagonist. In further embodiments, the stimulant antagonist is a benzodiazepine.

In one embodiment, the additional active ingredient(s) comprise an antidepressant. In further embodiments, the antidepressant is selected from any one or more of: agomelatine, Allegron (see nortriptyline), amitriptyline, Anafranil (see clomipramine), Brintellix (see vortioxetine), Cipralex (see escitalopram), Cipramil (see citalopram), citalopram, clomipramine, Cymbalta (see duloxetine), Depefex XL (see venlafaxine), dosulepin, doxepin, duloxetine, Edronax (see reboxetine), Efexor XL (see venlafaxine), escitalopram, Faverin (see fluvoxamine), fluoxetine, fluvoxamine, Foraven XL (see venlafaxine), imipramine, isocarboxazid, lofepramine, Lomont (see lofepramine), Lustral (see sertraline), Manerix (see moclobemide), mianserin, mirtazapine, moclobemide, Molipaxin (see trazodone), Nardil (see phenelzine), nortriptyline, Oxactin (see fluoxetine), Parnate (see tranylcypromine), paroxetine, phenelzine, Politid XL (see venlafaxine), Prothiaden (see dosulepin), Prozac (see fluoxetine), Prozep (see fluoxetine), reboxetine, Seroxat (see paroxetine), sertraline, Sinepin (see doxepin), Sunveniz XL (see venlafaxine), Surmontil (see trimipramine), Tofranil (see imipramine), Tonpular XL (see venlafaxine), tranylcypromine, trazodone, trimipramine, Triptafen, Valdoxan (see agomelatine), Venadex XL (see venlafaxine), Venaxx XL (see venlafaxine), venlafaxine, Venlalic XL (see venlafaxine), ViePax (see venlafaxine), vortioxetine, Zispin (see mirtazapine). In preferred embodiments, the antidepressant is selected from any one or more of: citalopram (Celexa), escitalopram (Lexapro), fluoxetine (Prozac), fluvoxamine (Luvox), paroxetine (Paxil), sertraline (Zoloft), desvenlafaxine (Pristiq), duloxetine (Cymbalta), levomilnacipran (Fetzima), milnacipran (Ixel, Savella), venlafaxine (Effexor), reboxetine (Edronax), teniloxazine (Lucelan, Metatone), or viloxazine (Vivalan).

In one embodiment, the additional active ingredient(s) comprise a nutraceutical. In further embodiments, the nutraceutical is selected from any one or more of: 5-methyltetrahydrofolic acid, ademetionine, adenine, adenosine monophosphate, alfacalcidol, alpha-linolenic acid, ATP, beta carotene, biotin, calcidiol, calcitriol, castor oil, cholecalciferol, choline, chondroitin sulfate, coenzyme A, coenzyme Q10, resveratrol, creatine, curcumin, cyanocobalamin, cystine, dihomo-gamma-linolenic acid, ephedra, ergocalciferol, eucalyptol, fish oil, folic acid, ginkgo biloba, ginkgolide-A, ginkgolide-B, ginkgolide-C, ginkgolide-J, ginkgolide-M, ginseng, ginsenoside C, ginsenoside Rb1, ginsenoside Rg1, glutamic acid, glutathione, glycine, glycine betaine, histidine, hyperforin, icosapent, icosapent ethyl, inulin, kava, krill oil, L-Alanine, L-Arginine, L-Asparagine, L-Aspartic Acid, L-Citrulline, L-Cysteine, L-Glutamine, L-Isoleucine, L-Leucine, L-Lysine, L-Phenylalanine, L-Proline, L-Threonine, L-Tryptophan, L-Tyrosine, L-Valine, lipoic acid, lutein, melatonin, menadione, methionine, N-Acetylglucosamine, NADH, niacin, octacosanol, omega-3 fatty acids, omega-6 fatty acids, ornithine, oxitriptan, oxogluric acid, pantothenic acid, phosphatidyl serine, phosphocreatine, prasterone, pyridoxal, pyridoxal phosphate, pyridoxine, pyruvic acid, riboflavin, sage oil, serine, serotonin, sesame oil, sinecatechins, spermine, St. John's Wort, succinic acid, taurine, tetrahydrofolic acid, thiamine, tretinoin, tyramine, ubidecarenone, ubiquinol, vitamin A, vitamin C, vitamin D, vitamin E, or vitamin K.

In additional embodiments, the additional active ingredient(s) comprise nicotine.

In another embodiment, the additional active ingredient(s) comprise a BCS Class II active ingredient. In further embodiments, the BCS Class II active ingredient is selected from any one or more of following: aceclofenac, albendazole, amiodarone, atorvastatin, azithromycin, bicalutamide, bisacodyl, cabergoline, candesartancilexetil, carbamazepine, carvedilol, cefditoren, celecoxib, chloroquine, chlorpromazine, cilostazol, ciprofloxacin, cisapride, clarithromycin, clofazimine, clopidogrel, clozapine, cyclosporine, cyproterone, danazol, dapsone, diazepam, diclofenac, diflunisal, digoxin, diloxanide, ebastine, efavirenz, epalrestat, eprosartan, erythromycin, ethylicosapentate, ezetimibe, fenofibrate, flurbiprofen, furosemide, gefitinib, gliclazide, glimepiride, glipizide, glyburide, glyburide(glibenclamide), griseofulvin, haloperidol, hydroxyzine, ibuprofen, imatinib, indinavir, indomethacin, irbesartan, isotretinoin, itraconazole, ketoconazole, ketoprofen, lamotrigine, lansoprazolei, lopinavir, loratadine, lorazepam, lovastatin, mebendazole, medroxyprogesterone, meloxicam, menatetrenone, metaxalone, metoclopramide, mosapride, mycophenolatemofetil, nabumetone, naproxen, nelfinavir, nevirapine, nicergoline, niclosamide, nifedipine, nimesulide, ofloxacin, olanzapine, orlistat, oxaprozin, phenazopyridine, phenytoin, pioglitazone, piroxicam, pranlukast, praziquantel, pyrantel, pyrimethamine, quetiapine, quinine, raloxifene, rebamipide, retinol, rifampicin, risperidone, ritonavir, rofecoxib, saquinavir, simvastatin, sirolimus, spironolactone, sulfasalazine, tacrolimus, talinolol, tamoxifen, telmisartan, teprenone, terfenadine, ticlopidine, tocopherolnicotinate, tosufloxacin, triflusal, ursodeoxycholicacid, valproicacid, valsartan, verapamil, warfarin, or zaltoprofen.

In another embodiment, at least one additional active ingredient is a BCS Class IV active ingredient. In further embodiments, the BCS Class IV active ingredient is selected from any one or more of following: acetaminophen (paracetamol), acetazolamide, acetylsalicylic acid, acyclovir, allopurinol, aluminium hydroxide, amoxicillin, azathioprine, cefdinir, cefixime, cefotiam, cefpodoxime, cefuroxime axetil, dapsone, dexamethasone, doxycycline, famotidine, folic acid, hydrochlorothiazide, l-carbocysteine, levodopa, linezolid, mesalamine, methylphenidate, metronidazole, modafinil, nalidixic acid, nitrofurantoin, nystatin, oxcarbazepine, oxycodone, phenobarbital, propylthiouracil, roxithromycin, sulfadiazine, sulfamethoxazole, sulpiride, sultamicillin, theophylline, or trimethoprim.

In one embodiment, the combined active ingredients in a composition of the present invention have synergistic activity, as compared to the additive activity of equivalent compositions comprising each active ingredient alone.

### Excipients

The aerosol formulation can further comprise additional physiologically or pharmaceutically acceptable excipients. Excipients can include, for example, solvents, flavoring agents, surfactants, preservatives, chelating agents, pH modifiers, humectants, valve lubricants, anti-oxidants, anti-aggregating agents, and fatty acids.

Flavor modifying excipients that may be added to the compositions described herein include peppermint oil, and menthol. An additional flavor modifying agent that can be added to the formulation that comprises ethanol and saline is saccharin and sodium saccharin. When the flavor modifying excipient is a solid, preferably it is micronized. The concentration will depend on the individual composition and the flavor modifying excipient. In some further embodiments, the composition further comprises: an amount of flavor modifying excipient selected from 0.01-0.025 wt %, 0.025-0.05 wt %, 0.05-0.1 wt %, 0.1-0.25 wt %, 0.25-0.5 wt %, 0.5-1 wt %, 1-2 wt %, 1-2.5 wt %, 1-5 wt %, 2-3 wt %, 3-4 wt %, 4-5 wt %, 5-7.5 wt %, 5-10 wt %, 10-12.5 wt %, 10-15 wt %, 10-20 wt %, 15-20 wt %, or 20-25 wt %, 25-30 wt %, or 25-50% wt %. In some further embodiments, the composition further comprises: an amount of sweetener selected from 0.01-0.025 wt %, 0.025-0.05 wt %, 0.05-0.1 wt %, 0.1-0.25 wt %, 0.25-0.5 wt %, 0.5-1 wt %, 1-2 wt %, 2-3 wt %, 2.5-5%, 3-4 wt %, 4-5 wt %, 5-6 wt %, 5-7.5 wt %, 7.5-10 wt %, or 5-10 wt %. As used herein, the term “flavoring” may represent a single species of flavor molecule (e.g., limonene) or a mixture of flavor molecule species (e.g., limonene, linalool, citral, citronellol, geranyl acetate and perillal) combined to produce a certain flavor.

In some embodiments, the composition comprises a surfactant that has an HLB value greater than 9, 10, 11, 12, 13, 14, 15, 16, or greater than 16. In other embodiments, the surfactant has an HLB value between 9-17, 9-16.7, 9-16, 9-15, 9-14, 10-17, 10-16.7, 10-16, 10-15, 12-14, 12-16, 14-16, 14-17, 15-17, and between 10-14.

In some embodiments, the surfactant is selected from: PEG 15 hydroxystearate (Solutol HS15), polyoxyl-10-Oleyl Ether (BRIJ® 97), polyethylene glycol 25 hydrogenated castor oil, polyethylene glycol (PEG) 40 hydrogenated castor oil (Kolliphor RH40, Cremophor RH40), polyethylene-polypropylene glycol (poloxamer 124), PEG 8 caprylic/capric glycerides (Labrasol), PEG 300 oleic glycerides (Labrafil M 1944), diethylene glycol monoethyl ether (Transcutol), lauroyl macrogol 32 glycerides (GELUCIRE® 44/14), polyethylene glycol 400 (PEG 400), propylene glycol laurate (Lauroglycol FCC), D-a-Tocopherol polyethylene glycol 1000 succinate (TPGS), polyethylene-polypropylene glycol (poloxamer 188), polyethylene-polypropylene glycol (poloxamer 407), polyvinyl pyrrolidone (e.g., Mw 28-34 kDa, Mw 44-54 kDa (e.g., Kollidon 30), or 1-1.5M kDa (e.g., Kollidon 90), Iota Carrageenan, Xanthan gum, locust Bean gum, Kelcogel LT100, acacia gum, guar gum, gamma-Cyclodextrin, Tracacanth gum, hydroxypropyl methylcellulose (HPMC), carboxymethyl cellulose (CMC), microcrystalline cellulose (MCC), lecithin, polyethylene-polypropylene glycol (poloxamer 124), polyethylene glycol sorbitan monolaurate (polysorbate 20, TWEEN 20), polyethylene glycol sorbitan monopalmitate (polysorbate 40, TWEEN 40), polyethylene glycol sorbitan monostearate (polysorbate 60, TWEEN 60), polyethylene glycol sorbitan tristearate (polysorbate 65, TWEEN 65), polyethylene glycol sorbitan monooleate (polysorbate 80, TWEEN 80), polyethylene glycol sorbitan trioleate (polysorbate 85, TWEEN 85), polyethylene glycol sorbitan hexaoleate, polyethylene glycol sorbitan tetraoleate, sorbitan monolaurate (Span 20), sorbitan monopalmitate (Span 40), sorbitan monostearate (Span 60), sorbitan tristearate (Span 65), sorbitane monooleate (Span 80), sorbitan trioleate (Span 85), sucrose laurate, sucrose palmitate, sucrose stearate, gamma-cyclodextrin, beta-cyclodextrin (e.g., CAPTISOL) pectin, whey protein, caseinates, quillaia/quillaj a saponins, quillaia extract, PEG 8 stearate, PEG 40 stearate, or a combination thereof.

In other embodiments, the surfactant is selected from: polyoxyl-10-Oleyl Ether (BRIJ® 97), polyethylene glycol 25 hydrogenated castor oil, polyethylene glycol (PEG) 40 hydrogenated castor oil (Kolliphor RH40, Cremophor RH40), polyethylene-polypropylene glycol (poloxamer 124), PEG 8 caprylic/capric glycerides (Labrasol), PEG 300 oleic glycerides (Labrafil M 1944), diethylene glycol monoethyl ether (Transcutol), sorbitane monooleate (Span 80), Lauroyl macrogol 32 glycerides (GELUCIRE® 44/14), polyethylene glycol 400 (PEG 400), propylene glycol laurate (Lauroglycol FCC), polysorbate 20 (TWEEN® 20), polysorbate 40 (TWEEN® 40), polysorbate 60 (TWEEN® 60), polysorbate 80 (TWEEN® 80), D-α-Tocopherol polyethylene glycol 1000 succinate (TPGS), polyethylene-polypropylene glycol (poloxamer 188), polyethylene-polypropylene glycol (poloxamer 407), polyvinyl pyrrolidone (Kollidon 30), polyvinyl pyrrolidone (Kollidon 90), Iota Carrageenan, Xanthan gum, locust Bean gum, Kelcogel LT100, acacia gum, guar gum, gamma-Cyclodextrin, Tracacanth gum, hydroxypropyl methylcellulose (HPMC), carboxymethyl cellulose (CMC), microcrystalline cellulose (MCC), lecithin, or a combination thereof.

In one embodiment, the composition comprises at least one fatty acid, at least one monoglyceride, at least one diglyceride, or at least one triglyceride, or a combination thereof. In one embodiment, the fatty acid, monoglyceride, diglyceride, triglyceride, or a combination thereof is an oil. In a further embodiment, the oil is selected from anise oil, apricot kernel oil PEG-6 esters, apricot kernel oil, beeswax, borage oil, canola oil, castor oil, polyoxyl 35 castor oil, polyoxyl 40 hydrogenated castor oil, polyoxyl 40 castor oil, polyoxyl 60 hydrogenated castor oil, hydrogenated castor oil, polyoxyl 60 castor oil, cinnamon oil, clove oil, coconut oil fractioned, coconut oil, coconut oil-lecithin, coriander oil, corn oil PEG-6 esters, corn oil PEG-8 esters, corn oil, cottonseed oil hydrogenated, cottonseed oil, cottonseed oil, hydrogenated soybean oil, hydrogenated vegetable oils, kernel oil PEG-6 esters, kernel oil, lemon oil, mineral oil (light), mineral oil, neutral oil, nutmeg oil, olive oil PEG-6 esters, olive oil, orange oil, palm kernel oil PEG-6 esters, palm kernel oil, palm kernel oil/palm kernel oil hydrogenated, palm fruit oil, peanut oil PEG-6 esters, peanut oil, peppermint oil, poppy seed oil, safflower oil, soybean oil hydrogenated, soybean oil refined, soybean oil, sunflower oil, triisostearin PEG-6 esters, vegetable oil hydrogenated, vegetable oil PEG esters, vegetable oil, vegetable oils glyceride hydrogenated, or a mixture thereof.

In one embodiment, the fatty acid, monoglyceride, diglyceride, triglyceride, or a combination thereof is a fat. In another embodiment, the fatty acid, monoglyceride, diglyceride, triglyceride, or a combination thereof is exogenously added fatty acid, monoglyceride, diglyceride, triglyceride, or a combination thereof. The term “exogenously added”, as used herein, means other than any fatty acids, monoglycerides, diglycerides, triglycerides, or combinations thereof, that were originally present in a cannabis plant, or other plant extract, and remains in the extract, e.g., a cannabinoid extract, after the extraction/distillation process. For clarity, pressed cannabis/hemp seed oil added to a composition of the present invention is exogenously added. In one embodiment, the only exogenously added fatty acid, monoglyceride, diglyceride, triglyceride, or a combination thereof, is a flavoring oil, e.g., flavor compounds diluted with and MCT or other oil. In a further embodiment, the flavoring oil is an essential oil. In a further embodiment, the essential oil is produced by distillation (e.g., steam distillation), solvent extraction (example, a hydrocarbon such as hexane or supercritical carbon dioxide), or by expression.

In another embodiment, the monoglyceride, diglyceride, or triglyceride is a medium chain monoglyceride, diglyceride, or triglyceride and/or a long chain monoglyceride, diglyceride triglyceride. In a further embodiment, the triglyceride is a medium chain triglyceride (MCT). In another further embodiment, the triglyceride is a long chain triglyceride (LCT).

The medium chain triglycerides (MCT) of the present invention are triglycerides whose fatty acids have an aliphatic tail of 6-12 carbon atoms. The MCT may be a single MCT or a mix of MCT. In one embodiment, the MCT is formed from fatty acids having from C6 to C8, C8 to C10, C10 to C12, or C8 to C12 carbon atoms. The fatty acids of the MCT may be saturated, mono-unsaturated, and/or poly-unsaturated fatty acids. In one embodiment 80 to 100% of the medium chain fatty acids are saturated, 0 to 10% are monounsaturated, and 0 to 5% are polyunsaturated. Preferred medium chain fatty acids include caproic acid, caprylic acid, capric acid, and mixtures thereof. An oil comprising MCT, may comprise at least 5 wt % medium chain triglycerides, e.g., coconut oil, or palm kernel oil. In one embodiment, the oil comprising an MCT is coconut oil. MCT may be in the form of oil that is enriched or fractionated to increase the concentration of medium chain triglycerides. In one embodiment, the MCT is fractionated coconut oil (e.g., glyceryl tricaprylate or NATURE'S OIL MCT). Medium chain triglycerides may also be formed by esterifying glycerol with mixtures of C6-C12 fatty acids, e.g., C8-C10 fatty acids such as caprylic (C:8) and capric (C:10) fatty acids fractionated from coconut or palm kernel oils.

The long chain triglycerides (LCT) of the present invention are triglycerides whose fatty acids have an aliphatic tail of 13-24 carbon atoms. The LCT may be a single LCT or a mix of MCT. In one embodiment, the LCT is formed from long chain fatty having from C14 to C16, C16 to C18, C18 to C20, C14 to C20, or C20 to C24 carbon atoms. The fatty acids of the LCT may be saturated, mono-unsaturated, and poly-unsaturated fatty acids. In one embodiment 5 to 25% of the long chain fatty acids are saturated, 15 to 80% are monounsaturated, and 15 to 80% are polyunsaturated. The oil comprising an LCT may comprise at least 5 wt % long chain triglycerides, e.g., olive oil, poppy seed, safflower, sunflower, corn, and soybean oils, sesame oil, or castor oil. LCT may be in the form of oil that is enriched or fractionated to increase the concentration of long chain triglycerides. In one embodiment, the LCT is olive oil.

The oil comprising an MCT and/or LCT may be selected from the group consisting of borage oil, castor oil, coconut oil, cottonseed oil, soybean oil, safflower oil, sunflower oil, castor oil, corn oil, olive oil, palm oil, peanut oil, poppy seed oil, canola oil, hydrogenated soybean oil, hydrogenated vegetable oils, sesame oil, triolein, trilinolein, and trilinolenin.

The compositions may include one or more antioxidant. Preferred antioxidants include ascorbic acid, ascorbyl palmitate, butylated hydroxy anisole, butylated hydroxy toluene, propyl gallate, α-tocopherol, γ-tocopherol, and mixed tocopherols. In one embodiment, the composition of the present invention further comprises an antioxidant(s) in the range of about 0.01% w/v to about 0.1% w/v. In some further embodiments, the composition further comprises an antioxidant(s) in the range of 0.1-0.25 wt %, 0.25-0.5 wt %, 0.5-1 wt %, 1-2 wt %, 2-3 wt %, 2.5-5%, 3-4 wt %, or 4-5 wt %.

A composition may comprise chelating agents in a final range of about 0.01% to about 0.5% w/v. Examples of chelating agents include ethylenediaminetetraacetic acid (EDTA), phosphoric acid, polyphosphates, polysaccharides, citric acid and combinations thereof.

A composition may also additionally comprise inactive ingredients selected from a group consisting of co-solvents, dispersing agents, emulsifiers, flavors, humectants, lubricants, preservatives, propellants, sorbents, suspension aids, sweeteners, tonicity modifiers, and combinations thereof.

A composition may further comprise a pH adjusting agent, e.g., disodium hydrogen phosphate, sodium acetate, sodium bicarbonate, sodium hydroxide, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and combinations thereof. In one embodiment, the composition pH is in the range of about 5.0 to about 6.5. In a further embodiment, the composition pH is in the range of about 6.5 to about 7.5. In a further embodiment, the composition pH is in the range of about 7.0 to about 7.5. In a further embodiment, the composition pH is in the range of about 6.5 to about 7.0.

A composition may additionally comprise an osmotic agent, e.g., glycerin, glucose, sucrose, sorbitol, sodium phosphate and combinations thereof.

A composition may further comprise a sweetener, flavoring and/or taste-masking agent, e.g., glucose, fructose, sucrose, sorbitol, sucralose, aspartame, neotame, acesulfame potassium, stevioside, sodium chloride, D-limonene, citric acid, xylitol and combinations thereof. In one embodiment, the sweetener is selected from one or more of: acesulfame potassium, advantame, aspartame, neotame, sucralose, stevia, glucose, fructose, sucrose, sorbitol, or xylitol. In one preferred embodiment, the sweetener is sucralose. An additional sweetener that can be added to the formulation that comprises ethanol and saline is saccharin and sodium saccharin.

A composition may also further comprise preservatives, e.g., methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, benzalkonium chloride and ethylene diamine tetra-acetic acid (EDTA), and combinations thereof.

### Methods of Treatment

The invention also encompasses methods of treating a subject having a disease or disorder that would benefit from the administration of a cannabinoid, comprising administering to said subject a therapeutically effective amount of the aerosol formulation comprising the cannabinoid by oral inhalation with a soft mist inhaler, wherein administration of said cannabinoid causes less coughing and/or upper and/or lower respiratory tract irritation than administration of said cannabinoid. Preferably, the subject is a human.

In one embodiment, the disease or disorder is selected from: Alzheimer Disease, Amyotrophic Lateral Sclerosis (ALS), pain, anxiety, nausea, vomiting, insomnia, restless leg syndrome (RLS), diabetes mellitus, dystonia, epilepsy, fibromyalgia, gastrointestinal disorders, inflammatory bowel disease, Crohn's disease, irritable bowel syndrome, gliomas, cancer, Hepatitis C, Human Immunodeficiency Virus (HIV) Huntington Disease, hypertension, incontinence, methicillin-resistant Staphyloccus aureus (MRSA), multiple sclerosis, osteoporosis, pruritus, rheumatoid arthritis, insomnia, sleep apnea, or Tourette Syndrome.

In one embodiment, the pain is chronic pain. In another embodiment, the pain is acute pain. In a further embodiment, the acute pain is a migraine. In a further embodiment, the pain is selected from any one or more of the following: post-herpetic neuralgia, trigeminal neuralgia, spinal cord injury pain, carpal tunnel syndrome, phantom limb, ischemic pain, pain resulting from sports injuries, back pain (e.g., low back pain), menstrual pain, gastrointestinal or urethral cramps, skin wounds, burns, or cancer pain. In a preferred embodiment, the pain is cancer pain.

In another embodiment, the nausea and/or vomiting results from a chemotherapy, e.g., cancer chemotherapy. In another embodiment, the nausea and/or vomiting results from opioid use.

In another embodiment, the method is for increasing socialization, increasing relaxation, inducing sleep, reducing the time needed to fall asleep, or for inducing a psychotropic effect (commonly known as a “high”). In another embodiment, the method is for reducing the amount of opioid(s) used by a subject suffering from pain or used by a subject addicted to an opioid.

The composition may be administered once, twice, three, or four times a day, or as needed.

In one embodiment, the invention provides a method of reducing the intensity or duration of pain in a subject (i.e., a subject, e.g., human), in need thereof, comprising the step of administering to the subject an effective amount of a cannabinoid composition of the present invention. In a further embodiment, the method decreases pain intensity in the subject. In a further embodiment, the method decreases pain duration in the subject. In one embodiment, the pain is acute pain. In another embodiment, the pain is chronic pain. In some embodiments, the subject has reduced pain intensity for at least 4 hours, at least 6 hours, at least 8 hours, at least 12 hours, at least 18 hours, or at least 24 hours post administration. In one embodiment, the cannabinoid composition of the present invention has a maximum pain-relieving effect between 1-4 hours or between 1.5-2.5 hours post administration. In another embodiment, the cannabinoid composition of the present invention has an onset of pain-relieving effect within 15 minutes, 20 minutes, 25 minutes, 30 minutes, or within 45 minutes post administration.

In one embodiment, the invention provides a method of reducing or preventing nausea or vomiting in a subject in need thereof, comprising administering to the subject an effective amount of a cannabinoid composition of the present invention. In one embodiment, the nausea or vomiting is opioid induced nausea or vomiting. The opioid inducing the nausea or vomiting may be an opioid analgesic such as hydrocodone, oxycodone, oripavine, dihydromorphine, hydromorphinol, nicomorphine, dipropanoylmorphine, diacetyldihydromorphine, desomorphine, methyldesorphine, heterocodeine, benzylmorphine, dihydroheterocodeine, myrophine, pentamorphone, tramadol, fentanyl, etc. In one embodiment, the cannabinoid composition is administered 0-30 minutes, 30-60 minutes prior to administration of the opioid. In another embodiment, the cannabinoid composition is administered 60 minutes prior to administration of the opioid. In another embodiment, the cannabinoid composition is administered concurrently with the administration of the opioid. In one embodiment, the nausea or vomiting occurs after surgery and results from anesthesia.

In one embodiment, the subject has reduced intensity of nausea in the 2 hours, 3 hours, 4 hours, 5 hours, 6 hours, 8 hours, 12 hours, 18 hours, or 24 hours following initial administration of the cannabinoid containing composition. In one embodiment, the subject has reduced vomiting in the 4 hours, 6 hours, 8 hours, 12 hours, 18 hours, or 24 hours following initial administration of the cannabinoid composition. In one embodiment, the cannabinoid composition of the present invention has a maximum nausea or vomiting reducing effect between 1-4 hours, 1-3 hours, 2-4 hours, or between 1.5-2.5 hours post administration. In another embodiment, the cannabinoid composition of the present invention has an onset of nausea or vomiting reducing effect within 15 minutes, 20 minutes, 25 minutes, 30 minutes, or within 45 minutes post administration.

In one embodiment, the method of reducing nausea or vomiting in a subject includes reducing the occurrence of nausea or vomiting.

The invention is illustrated by the following non-limiting examples.

### EXEMPLIFICATION

### Example 1: Viscosity and FPF Characterization of Ethanol-Based THC and CBD Formulations

### Preparation of Ethanol-Based Formulations

Δ9-THC distillate was heated in a 60° C. oven for 30 minutes. Subsequently, 10.2 g of distillate and 16.4 g of 200 proof ethanol were transferred via pouring to a clean, dry 2-ounce jar. The jar was sealed and mixed at room temperature using a Multi-Tube Vortexer (VWR DVX-2500). Upon fully dissolving, the resulting distillate-ethanol tincture had a distillate concentration of 38%w/w. Serial dilutions of 19, 9.6, 4.8, 2.4, and 1.2% w/w distillate in 200 proof ethanol were prepared by diluting 10 g of formulation with 10 g of 200 proof ethanol. Samples were stored at 4° C. until use.

This process was followed for the preparation of ethanol-based formulations containing CBD isolate with the exception that the CBD isolate was weighed into the clean, dry 2-ounce jar using a spatula (no pre-heating at 60° C. required). Solution viscosities were measured using a RheoSense microVISC™ rheometer.

Formulations containing saline were prepared by substituting ethanol with normal saline (0.9%w/v NaCl in water) to achieve 5%, 10%, 20%, 30%, 40%, or 50%w/w saline in the final formulation.

### Fine Particle Fraction (FPF)

An abbreviated impactor set-up, Copley Scientific's ‘Fast Screening Impactor’ (FSI), was used to determine the FPF within liquid aerosols (Mitchell et al. AAPS PharmSciTech. 2009;10(1):252-7). The FSI consisted of the universal induction port (UIP), course fraction collector (pre-separator and insert with a 5 μm diameter cut-off @ 28.3 L/min), and fine particle fraction collector consisting of a holder and glass fiber filter. The system was assembled along with a Copley LC5 pump. The flow rate was set by attaching a flow meter to the inlet of the induction port and adjusting the flow control valve on the LC5 vacuum pump until the flow rate through the system was 28.3 L/min +/−5%. Measurements were performed under ambient laboratory conditions (˜60% RH, 72° F.). The Respimat device was primed and attached to the UIP using a mouthpiece adapter. With the vacuum pump running, the device was actuated followed by a 30 second hold before turning off the vacuum pump. The Respimat device was re-primed and another dose was collected in the FSI. To determine the amount of cannabinoids deposited in each stage, the FSI was dismantled and each stage was extracted with 10 ml of methanol. Samples were diluted and HPLC performed to quantify the mass of cannabinoids deposited in the USP throat, coarse fraction collector, and fine particle fraction collector.

The aerosol size distribution was determined using a laser diffraction system (Spraytec; Malvern Instruments Inc., Southborough, Mass.) outfitted with an inhalation cell that enables measurements to be performed under controlled flow rate. The inhalation cell was positioned in the horizontal set-up in order to measure the size distribution of the aerosol directly after it is emitted from the inhaler device.

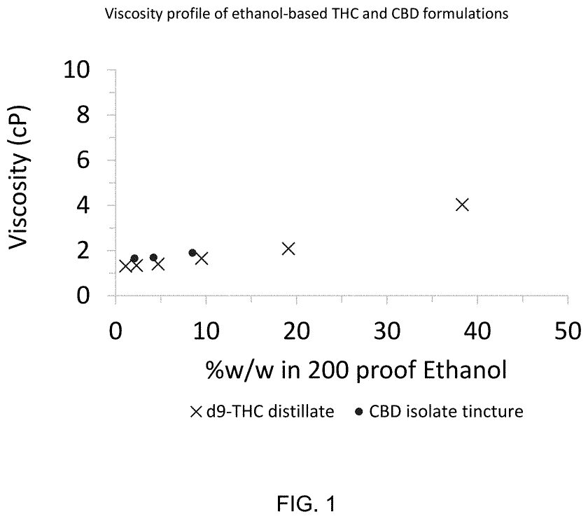

The solution viscosity, along with surface tension and solvent volatility, play a role in determining aerosol particle size. FIG. 1 shows a plot of viscosity (cP) as a function of the percentage (%) w/w of Δ9-THC distillate, CBD isolate in 200 proof ethanol.

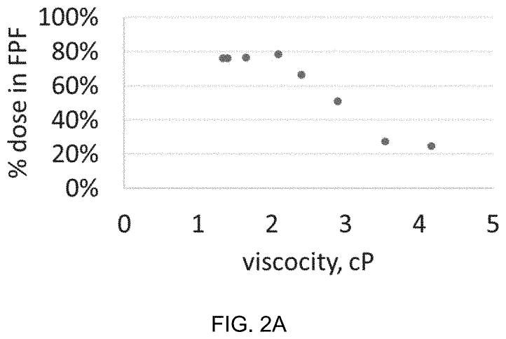

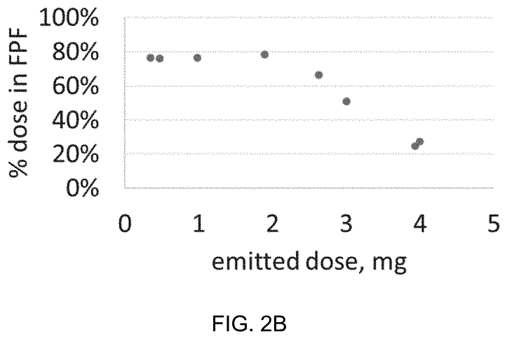

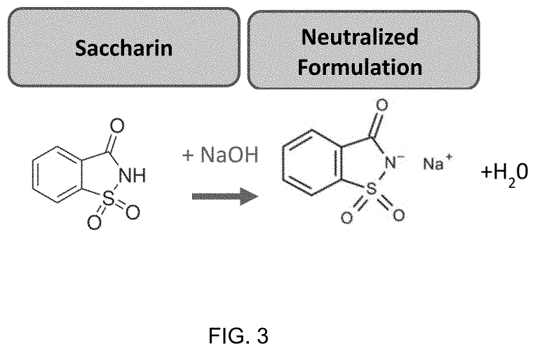

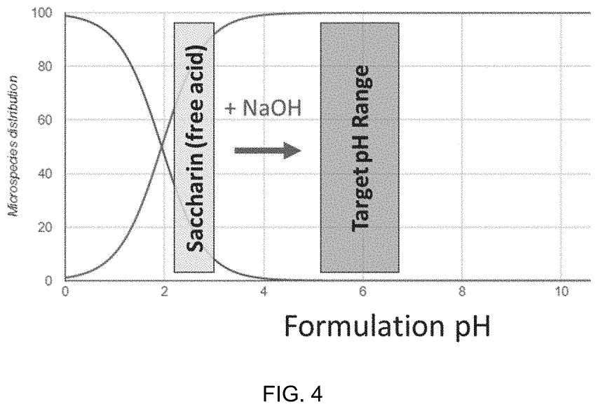

FIG. 2A and 2B are plots of percent dose of Δ9-THC in the fine particle fraction (FPF) as a function of viscosity (cP) (FIG. 2A) and emitted dose (mg) (FIG. 2B). As shown in these figures, formulations with viscosity less than 2 cP and/or less than 2 mg emitted dose delivered about 80% of the dose in FPF.

### Example 2: Observation Feedback of the Tolerability of Δ9-THC Distillate in Ethanol Delivered via RESPIMAT®

Volunteers reported that 200 proof ethanol (placebo) delivered using the RESPIMAT® device as non-irritating. It was observed that the cannabinoid, Δ9-THC in the ethanol solution, induced irritation that was dose dependent as shown in Table 1 below:

TABLE 1

| Dose | | % dose |
|---|---|---|
| (mg) | User experience | in FPF |
| 2 | Extremely harsh: Coughing with throat/upper airway | 78% |
| | irritation that persists greater than 5 minutes. | |
| 1 | Harsh: Coughing with throat/upper airway irritation | 77% |
| | that dissipates within 5 minutes. | |
| 0.6 | Harsh: Mild cough with mild throat/upper airway | 76% |
| | irritation that dissipates within 5 minutes. | |
| 0.35 | Most tolerable: Cough may occur, mild irritation of | 76% |
| | throat/upper airways that dissipates within 2 minutes. | |

The irritation was caused by cannabinoid deposition at mucosal membranes in the throat and upper airways. The lung deposition profile is affected by multiple factor including FPF as well as user inhalation technique. For example, it has been shown that the timing of inhalation and actuation as well as the posture of the user's head can have an impact on lung deposition. It is also important to note that in vitro impactor studies commonly underestimate dose deposition in the throat and overestimate the dose deposited in the lungs (Wei et al. (2018), Journal of Aerosol Medicine and Pulmonary Drug Delivery 31(0): 1-14).

Even using a low dose of cannabinoid and a high performing device like RESPIMAT®, it is difficult to avoid cannabinoid deposition in the throat/upper airway. It is anticipated that improper user technique would further intensify the level of irritation.

### Example 3: Administration of Saccharin and Sodium Saccharin Containing Formulations

It was observed that administration of saccharin containing formulations induced minimal to no throat irritation as compared to cannabinoid formulations that did not contain saccharin. While saccharin-containing aerosol formulations reduced coughing and throat irritation, many users experienced chest discomfort or chest tightness after inhalation that lasted approximately 30 minutes or longer. The saccharin-containing formulation (containing 0.1% w/w saccharin as a free acid) has a pH of about 3 and it has been discovered that an aerosol formulation that has a pH closer to neutral is better tolerated. Specifically, administration of a formulation containing 0.1% w/w sodium saccharin in place of saccharin (free acid), and that had a pH of about 6.5, resulted in less chest discomfort or tightness as compared to the saccharin (free acid)-containing formulation. Overwhelmingly, users prefer the neutral sodium saccharin containing formulations over formulations containing saccharin as a free acid. Furthermore, the inclusion of saccharin in the Δ9-THC formulation had no impact on the lung deposition profile of the formulation and potency was maintained.

TABLE 2
Exemplary formulations comprising saccharin:

| | Δ9- | | | | | |
|---|---|---|---|---|---|---|
| | THC | | | | | |
| Formu- | distillate, | Saccharin, | Menthol, | Ethanol, | Saline, | |
| lation | % w/w | % w/w | % w/w | % w/w | % w/w | pH |
| A | 2 | 0.1 | 0 | 67.9 | 30 | 3 |
| B | 8 | 0.1 | 0.025 | 81.9 | 10 | 3 |
| C | 8 | 0.1 | 0.025 | 81.9 | 10 | 6.5 |
| D | 4 | 0.1 | 0.025 | 85.9 | 10 | 6.5 |
| E | 4 | 0.1 | 0 | 95.9 | 0 | 6.4 |

Formulation C containing approximately 0.1% sodium saccharin was prepared by adding an equimolar amount of sodium hydroxide to Formulation B to convert the acidic saccharin to neutral sodium salt form. The pH of Formulation C was 6.5.

Sodium saccharin containing formulations can also be prepared by adding sodium saccharin to solution of Δ9-THC distillate in ethanol. The formulation can further contain saline and/or menthol.

### Example 4: Effect of Saline Addition to Ethanol-Containing Cannabinoid Aerosol Formulations

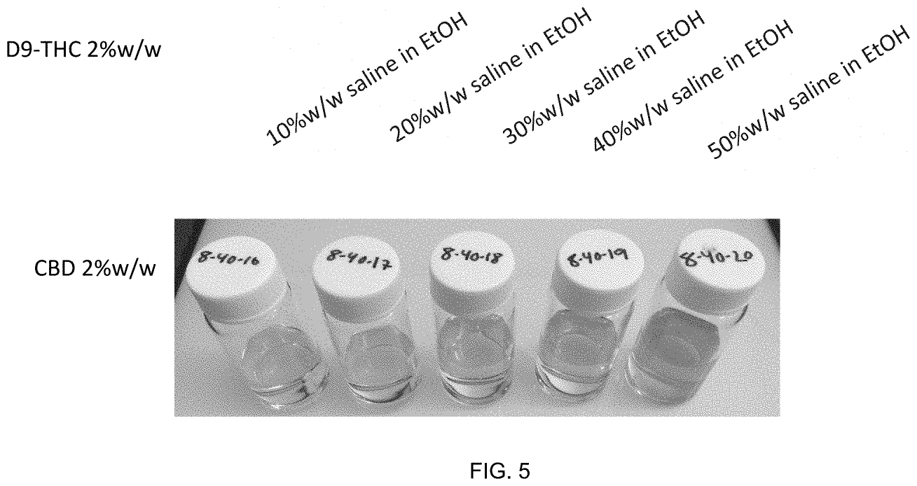

As shown in FIG. 5 the solubility of Δ9-THC and CBD in a solution of ethanol and saline was investigated. A 2% w/w solution of Δ9-THC was prepared at room temperature in solutions of 10%, 20%, 30%, 40%, or 50% (w/w) saline in ethanol. 2% w/w Δ9-THC was soluble at room temperature in ethanol solutions of up to 30% saline. A 2% w/w solution of CBD was prepared at room temperature in solutions of 10%, 20%, 30%, 40%, or 50% (w/w) saline in ethanol. 2% w/w CBD was soluble at room temperature in ethanol solutions of up to 40% saline. In a separate experiment, the absolute solubility of Δ9-THC in ethanol solutions containing 30%w/w saline was determined to be ˜8%w/w (data not shown).

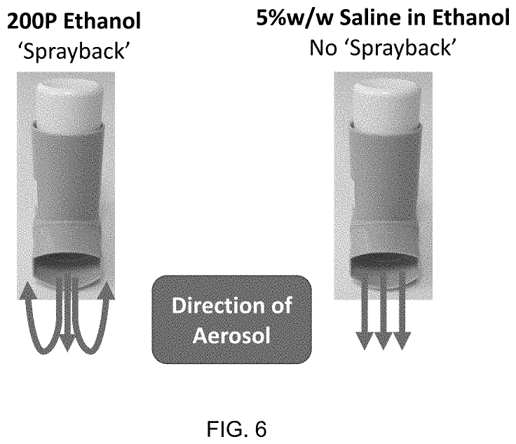

It was observed that the 200 proof ethanol solutions in the Medspray inhaler resulted in “sprayback” as illustrated in FIG. 6. It was believed that electrostatics caused the aerosol to deposit within the mouthpiece of the device thus reducing the emitted dose. The conductivity of the solution was increased with 5% saline which reduced aerosol charging and increased the emitted dose. Table 3 below shows the conductivity measurements for ethanol solutions comprising 0%, 5%, and 10% saline.

TABLE 3

| | Conductivity |
|---|---|
| % Saline | μS/cm |
| 0% | 1 |
| 5% | 228 |
| 10% | 435 |

The saline-containing formulations were also tested in the Fast Screening Impactor set up described above and the data is shown below in Table 4:

TABLE 4

| | | Measurement 1 | Measurement 2 |
|---|---|---|---|
| Formulation description | FSI position | % dose | % dose |
| 8% distillate, 0% saline | Inlet (throat) | 10% | 9% |
| | coarse | 13% | 11% |
| | fine fraction | 77% | 79% |
| 8% distillate, 5% saline | Inlet (throat) | 6% | 3% |
| | coarse | 5% | 6% |
| | fine fraction | 90% | 91% |
| 8% distillate, 10% saline | Inlet (throat) | 4% | 3% |
| | coarse | 5% | 8% |
| | fine fraction | 91% | 89% |
| 8% distillate, 20% saline | Inlet (throat) | 6% | 4% |
| | coarse | 4% | 9% |
| | fine fraction | 90% | 87% |
| 8% distillate, 30% saline | Inlet (throat) | 5% | 10% |
| | coarse | 5% | 9% |
| | fine fraction | 90% | 81% |

As shown in Table 4, the percentage of the dose in the coarse particle fraction was 11 and 13% and the percentage of the dose in the inlet (throat fraction) was 9 and 10% for the formulation comprising no saline during two separate measurements. In contrast, for the 5% saline formulation, the coarse particle fraction was 5 and 6% and the percentage of the dose in the inlet was 3 and 6%. For the 10% saline solution, the coarse particle fraction was 5 and 8% and the percentage of the dose in the inlet was 3 and 4%. Thus, addition of saline improved the aerosol performance by reducing the fraction of aerosolized distillate that deposited in the inlet (throat). Similar performance was achieved with formulations containing 20% saline. As saline content increased further to 30%, the percentage of the dose in the inlet increased slightly to 5 and 10%. Therefore, formulations containing 5, 10, and 20% saline appeared superior with respect to percentage of large particles than the 30% saline solutions.

Laser diffraction data (volume-based particle size) is shown below in Table 5:

TABLE 5

| Formulation description | Dv(90) μm |
|---|---|
| 8% distillate, 0% saline | 5.17 |
| 8% distillate, 5% saline | 6.27 |
| 8% distillate, 10% saline | 7.28 |
| 8% distillate, 20% saline | 8.59 |
| 8% distillate, 30% saline | 8.22 |

As shown in Table 5, the Dv(90) particle size (particle diameter below which 90% of the aerosol volume exists) appears to increase with increasing saline content. Aerosol formulations containing ethanol benefit from partial evaporation of the ethanol resulting in reduced aerosol particle diameters. The trend of increasing particle size with increasing saline content may be attributed to the reduced volatility of saline as compared to ethanol.

Two specific examples of saline-containing formulations are delineated in Tables 6 and 7 below. The formulations comprising Δ9-THC in an ethanol solution comprising 10% saline, wherein the formulation further comprises 0.1% w/w saccharin and 0.025% w/w menthol. The osmolarity of the two formulations is also shown in the Tables:

TABLE 6
Formulation A

| Description | % w/w | mOsm |
|---|---|---|
| D9-THC | 8% | 211 |
| NaCl | 10% Saline (0.9% w/v NaCl) | 26 |
| Saccharin | 0.1% | 5 |
| Menthol | 0.025% | 1 |

TABLE 7
Formulation B

| Description | % w/w | mOsm |
|---|---|---|
| D9-THC | 8% | 211 |
| NaCl | 10% Saline (4% w/v NaCl) | 114 |
| Saccharin | 0.1% | 5 |
| Menthol | 0.025% | 1 |

The total osmolarity of Formulation A (Table 6) is 242 mOsm and the total osmolarity of Formulation B (Table 7) is 325 mOsm.

### Example 5: Terpene Solubility Assessment in 10% Saline, 20% Water, 70% Ethanol

The solubility of individual terpenes and a custom terpene blend was assessed in a base formulation consisting of 10% Saline, 20% Water and 70% Ethanol. The base formulation was prepared in bulk and 15 g aliquots were transferred to 22 ml scintillation vials. Pure, botanically derived terpenes were purchased from commercial sources. A custom terpene blend of five terpenes that are commonly found in cannabis was prepared and consisted of the following composition: 40% w/w β-caryophyllene, 15% w/w α-pinene, 15% w/w beta-pinene, 10% w/w Eucalyptol, and 20% w/w Limonene. Individual terpenes or the custom terpene blend were spiked into the base formulation using positive displacement pipet and solutions mixed overnight on benchtop rotary shaker at room temperature before recording visual observations.

Terpenes including Linalool, Terpinolene, β-caryophyllene, α-pinene, β-pinene, Eucalyptol and Limonene and the custom terpene blend were soluble at concentrations of 0.12% w/w or less (Table 8; myrcene presented solubility challenges and was not soluble even at the lowest percentage tested (0.06% w/w).

TABLE 8
Solubility assessment of individual terpenes and a custom terpene blend
in base formulation containing 10% Saline, 20% Water, 70% Ethanol.

| | Target | |
|---|---|---|
| | Concentration, | Visual |
| Terpene/Terpene Mixture | % w/w | Observation |
| β-myrcene | 0.24% | Hazy, not fully |
| | | soluble |
| | 0.12% | Hazy, not fully |
| | | soluble |
| | 0.06% | Hazy, not fully |
| | | soluble |
| Linalool | 0.24% | Hazy, not fully |
| | | soluble |
| | 0.12% | Clear, colorless |
| | 0.06% | Clear, colorless |
| Terpinolene | 0.24% | Hazy, not fully |
| | | soluble |
| | 0.12% | Clear, colorless |
| | 0.06% | Clear, colorless |
| β-caryophyllene | 0.24% | Hazy, not fully |
| | | soluble |
| | 0.12% | Clear, colorless |
| | 0.06% | Clear, colorless |
| α-pinene | 0.24% | Hazy, not fully |
| | | soluble |
| | 0.12% | Clear, colorless |
| | 0.06% | Clear, colorless |
| β-pinene | 0.24% | Hazy, not fully |
| | | soluble |
| | 0.12% | Clear, colorless |
| | 0.06% | Clear, colorless |
| Eucalyptol | 0.24% | Hazy, not fully |
| | | soluble |
| | 0.12% | Clear, colorless |
| | 0.06% | Clear, colorless |
| Limonene | 0.24% | Hazy, not fully |
| | | soluble |
| | 0.12% | Clear, colorless |
| | 0.06% | Clear, colorless |
| Custom Terpene Blend: | 0.24% | Hazy, not fully |
| 40% w/w β-caryophyllene, 15% w/w | | soluble |
| α-pinene, 15% w/w β-pinene, | 0.12% | Clear, colorless |
| 10% w/w Eucalyptol, and 20% w/w | | |
| Limonene | 0.06% | Clear, colorless |

### Example 6: Characteristics of Representative THC and CBD-Based Formulations

Formulations E73, E74, E75, E76, E81, E82, E83, E84, E88 and E89 were prepared containing THC distillate (in amounts between 4 and 10% w/w) in 10% saline in ethanol (in amounts between 79.9 to 86% w/w), and other components (sodium saccharin, menthol, and the custom terpene blend described in Example 5) in the amounts shown in Tables 9. The formulations were all clear and light amber in color. Table 9 also shows the measured density, viscosity and pH of the formulations.

TABLE 9
Representative THC Formulations containing 10% saline in ethanol.

| | THC | | | | Na | | | | | |
|---|---|---|---|---|---|---|---|---|---|---|
| | Distillate | Saline | Water | Ethanol | Saccharin | Other | Visual | Density, | Viscosity, | |
| ID | % w/w | % w/w | % w/w | % w/w | % w/w | % w/w | Appearance | g/ml | cP | pH |
| E88 | 10% | 10% | 0% | 79.9% | 0.1% | | Clear, | 0.84 | 2.2 | 6.4 |
| E73 | 8% | 10% | 0% | 81.9% | 0.1% | 0.025% | light | 0.84 | 2.2 | 6.5 |
| | | | | | | Menthol | amber | | | |
| E74 | 4% | 10% | 0% | 85.9% | 0.1% | | | 0.83 | 2.2 | 6.5 |
| E75 | 8% | 10% | 0% | 82% | | | | 0.83 | 2.2 | 6.4 |
| E76 | 4% | 10% | 0% | 86% | | | | 0.82 | 2.0 | 6.4 |
| E81 | 8% | 10% | 0% | 81.9% | 0.1% | | | 0.83 | 2.3 | 6.3 |
| E82 | 4% | 10% | 0% | 85.9% | 0.1% | | | 0.82 | 2.1 | 6.5 |
| E83 | 8% | 10% | 0% | 82% | | | | 0.83 | 2.0 | 6.3 |
| E84 | 4% | 10% | 0% | 86% | | | | 0.82 | 2.2 | 6.5 |
| E89 | 8% | 10% | 0% | 82% | 0.1% | 0.12% | | 0.83 | 2.1 | 6.4 |
| | | | | | | Custom | | | | |
| | | | | | | Terpene | | | | |
| | | | | | | Blend | | | | |

For preparation of CBD-based formulations similar to the THC formulations described in Table 9, CBD isolate can be successfully substituted for the THC distillate.

Formulations E91, E92, E93 and E94 were prepared containing CBD isolates (in amounts between 2 and 10% w/w) in 10% saline and 20% water in ethanol (in amounts between 60 to 68% w/w) as described in Table 10. Formulation E91 was opaque and the presence of precipitates was evident. Formulations E92, E93 and E94 were clear and colorless. Table 9 also shows the measured density, viscosity and pH of the formulations.

TABLE 10
Representative CBD Formulations containing 10% saline and 20% water
in ethanol. For preparation of THC-based formulations

| | CBD | | | | | | | |
|---|---|---|---|---|---|---|---|---|
| | Isolate | Saline | Water | Ethanol | Visual | Density, | Viscosity, | |
| ID | % w/w | % w/w | % w/w | % w/w | Appearance | g/ml | cP | pH |
| E91 | 10% | 10% | 20% | 60% | Opaque, | n/a | n/a | n/a |
| | | | | | precipitates | | | |
| | | | | | present | | | |
| E92 | 8% | 10% | 20% | 62% | Clear, | 0.88 | 2.8 | 6.1 |
| E93 | 4% | 10% | 20% | 66% | colorless | 0.87 | 2.8 | 6.1 |
| E94 | 2% | 10% | 20% | 68% | | 0.88 | 2.8 | 6.3 |

For preparation of THC-containing formulations similar to the CBD formulations described in Table 10, THC distillate can be successfully substituted for the CBD isolate.

### Example 6: THC Dose Escalation w/MEDSPRAY® Inhaler Device (MIST Beta, Low Airflow Rate)

Users who used the MEDSPRAY® Inhaler device (MIST Beta, low airflow rate) with formulations of varying potency (THC content) provided feedback on how their experiences varied with the THC dose. Devices were loaded with 4 separate formulations containing 2, 4, 6, or 8% THC distillate. See Table 11. All formulations contained 10% saline and 20% water to reduce potential for unwanted taste/irritation caused by formulations containing high ethanol content (80% or higher). Users preferred E44 due to highest potency (8% THC distillate) which delivers noticeable effects within 2-3 puffs. All formulations delivered using MEDSPRAY® Inhaler device (low flow rate) induced some level of mild coughing and/or irritation of the throat/upper airways. Users also reported an unpleasant bitter taste.

TABLE 11
Summary of formulations delivered by MEDSPRAY ® Inhaler device
(MIST beta, low airflow rate) and resulting device performance emitted
including THC dose and % emitted dose in the fine particle fraction (%
FPF) as measured at 15 liters/min (LPM).

| | | Active | | Emitted | |
|---|---|---|---|---|---|
| | | Ingredient | | THC | % Dose |
| Formulation | | and | Other | dose, | in FPF |
| ID | Device | Content | Ingredients | mg/puff | (15 LPM) |
| E38 | MIST Beta | 2% THC | 10% Saline, | 0.3 | 78% |
| | | Distillate | 20% Water | | |
| | | | 68% | | |
| | | | Ethanol | | |
| E40 | MIST Beta | 4% THC | 10% Saline, | 0.6 | 79% |
| | | Distillate | 20% Water, | | |
| | | | 66% | | |
| | | | Ethanol | | |
| E41 | MIST Beta | 6% THC | 10% Saline, | 0.8 | 77% |
| | | Distillate | 20% Water, | | |
| | | | 64% | | |
| | | | Ethanol | | |
| E44 | MIST Beta | 8% THC | 10% Saline, | 1.1 | 76% |
| | | Distillate | 20% Water, | | |
| | | | 62% | | |
| | | | Ethanol | | |

### REFERENCES

1. Williams, S., J. Hartley, and J. Graham, Bronchodilator effect of delta1-tetrahydrocannabinol administered by aerosol of asthmatic patients. Thorax, 1976. 31(6): p. 720-723.
2. Tashkin, D. P., et al., Bronchial effects of aerosolized Δ9-tetrahydrocannabinol in healthy and asthmatic subjects. American Review of Respiratory Disease, 1977. 115(1): p. 57-65.
3. Naef, M., et al., Development and pharmacokinetic characterization of pulmonal and intravenous delta-9-tetrahydrocannabinol (THC) in humans. Journal of pharmaceutical sciences, 2004. 93(5): p. 1176-1184.
4. Polverino, M., et al., Anatomy and neuro-pathophysiology of the cough reflex arc. Multidisciplinary respiratory medicine, 2012. 7(1): p. 5.
5. Meyer, Pascale & Langos, Manuela & Brenneisen, Rudolf (2018). Human Pharmacokinetics and Adverse Effects of Pulmonary and Intravenous THC-CBD Formulations. Medical Cannabis and Cannabinoids. 1. 36-43. 10.1159/000489034.

All references, articles, patent applications, patent publications and patents are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

1. A method for reducing cannabinoid-related irritation and/or coughing of an aerosol formulation comprising a cannabinoid, the method comprising:
adding an irritation-reducing amount of saccharin or a salt thereof to an aerosol formulation comprising ethanol and a cannabinoid;
loading a soft mist inhaler with the aerosol formulation; and
administering the aerosol formulation to a subject by oral inhalation using the soft mist inhaler.

2. (canceled)

3. The method of claim 1, wherein the formulation does not comprise a propellant.

4. The method of claim 1, wherein the formulation does not comprise a glycol or a glycol ether.

5. (canceled)

6. The method of claim 1, wherein the amount of saccharin or the salt thereof is between about 0.08% to about 0.2% w/w.

7. (canceled)

8. The method of claim 1, wherein the formulation comprises the salt of saccharin.

9. (canceled)

10. The method of claim 8, wherein the saccharin salt is sodium saccharin.

11. The method of claim 10, wherein the sodium saccharin is an amount between about 0.08% to about 0.2% w/w.

12. The method of claim 10, wherein the pH of the formulation is between about 5 and 7.5.

13. The method of claim 1, wherein the amount of ethanol in the formulation is between about 60 and about 97% w/w.

14. The method of claim 1, wherein the formulation further comprises menthol.

15. The method of claim 1, wherein the solution comprising ethanol further comprises saline.

16. The method of claim 1, wherein the cannabinoid is selected from the group consisting of CBD, Δ9-tetrahydrocannabinol (Δ9-THC), Δ8-tetrahydrocannabinol (Δ8-THC), CBG, THCV, CBN, CBC, and CBDV or a combination thereof.

17. (canceled)

18. The method of claim 16, wherein the cannabinoid is Δ9-THC or CBD, or a combination thereof.

19. (canceled)

20. (canceled)

21. The method of claim 1, wherein the formulation comprises:
a. Δ9-THC or CBD in an amount between about 2 and about 10% w/w;
b. saccharin or the salt thereof in an amount of about 0.08 to about 0.2% w/w;
c. ethanol in an amount between about 60 and about 85%; and
d. water in an amount between about 0 and about 25%.

22. (canceled)

23. The method of claim 21, wherein the saccharin or the salt thereof is sodium saccharin and the pH of the formulation is between 5.5 and 7, optionally between 6 and 7.

24. The method of claim 21, wherein the saccharin or the salt thereof is sodium saccharin.

25-30. (canceled)

31. An aerosol formulation for pulmonary administration of a cannabinoid by oral inhalation using a soft mist inhaler,
wherein the aerosol formulation comprises the cannabinoid in a solution comprising ethanol, and wherein the formulation further comprises saccharin or a salt thereof,
wherein the administration of the aerosol formulation comprising the cannabinoid induces less coughing, less throat irritation, and/or less respiratory tract irritation than the administration of an identical formulation in the absence of the saccharin or salt thereof, and wherein the formulation does not comprise a propellant or a glycol or a glycol ether.

32. (canceled)

33. A method for reducing cannabinoid-related irritation and/or coughing of an aerosol formulation comprising a cannabinoid, the method comprising adding an irritation-reducing amount of saline to an aerosol formulation comprising ethanol and a cannabinoid, loading a soft mist inhaler with the aerosol formulation, and administering the aerosol formulation to a subject by oral inhalation using the soft mist inhaler.

34. (canceled)

35. (canceled)

36. The method of claim 33, wherein the solution comprises saline in amount of about 1% to about 30% by weight.

37. (canceled)

38. (canceled)

39. The method of claim 36, wherein the solution comprises saline in an amount of about 5 to about 10% by weight.

40. (canceled)

41. The method of claim 33, wherein the solution comprises the cannabinoid in an amount of about 5% to about 10% w/w.

42. The method of claim 33, wherein the fine particle fraction of the emitted dose is at least about 80%.

43-51. (canceled)

52. The method of claim 33, wherein the cannabinoid is selected from the group consisting of CBD, Δ9-THC, Δ8-THC, CBG, THCV, CBN, CBC, and CBDV or a combination thereof.

53-66. (canceled)
